(19) United States
(12) Patent Application Publication
(10) Pub. No.: US 2012/0295930 A1
(43) Pub. Date: Nov. 22, 2012

(54) NOVEL PROCESS FOR THE PREPARATION OF CIS-NUCLEOSIDE DERIVATIVE

(76) Inventors: Shankar Rama, Hyderabad (IN); Sarat Chandra Srikanth Gorantla, Hyderabad (IN); Lakshmana Rao Vadali, Hyderabad (IN); Venkata Bala Kishore Sarma Inupakutika, Hyderabad (IN); Srinivas Rao Dasari, Hyderabad (IN); Nagaraju Mittapelly, Hyderabad (IN); Santosh Kumar Singh, Hyderabad (IN); Debashish Datta, Hyderabad (IN)

(21) Appl. No.: 13/577,118

(22) PCT Filed: Feb. 1, 2011
(86) PCT No.: PCT/IN11/00070

§ 371 Date: Aug. 3, 2012

(30) Foreign Application Priority Data
Feb. 3, 2010 (IN) 264/CHE/2010
Feb. 15, 2010 (IN) 358/CHE/2010
Feb. 26, 2010 (IN) 2470/CHE/2010
Aug. 4, 2010 (IN) 940/CHE/2010

(51) Int. Cl. C07D 411/04; A61P 31/18; A61P 31/14; C07D 411/12; A61K 31/513

(52) U.S. Cl. 514/274; 544/317; 546/280.4

(57) ABSTRACT

The present invention relates to an improved process for the preparation of cis-nucleoside derivative of formula-1 involving chlorination of the compound of formula-2 followed by reaction with compound of formula-3 in presence of a base to get compound of formula-4, reacting the compound of formula-4 with an alkyl halide (RiX) to get a quaternary ammonium salt then with cytosine derivative of formula-5 to provide the compound of formula-6, optionally de-protecting the compound of formula-6 to the compound of formula-7, reducing compound of formula-7 with metal catalyst in presence of a buffer solution, then adding an organic acid to get the compound of formula-8, and converting the compound of formula-8 to cis-nucleoside derivative of formula-1. The present invention further relates to novel cis-nucleoside derivative of formula-8. The present invention also relates to a pharmaceutical composition comprising cis-nucleoside derivative of formula-1 with excipients.

This application claims priority to Indian patent applications 264/CHE/2010 filed on Feb. 3, 2010; 358/CHE/2010 filed on Feb. 15, 2010; 940/CHE/2010 filed on Apr. 5, 2010 2470/CHE/2010 filed on Aug. 26, 2010 the contents of which are incorporated by reference in their entirety.

### FIELD OF THE INVENTION

The present invention relates to a novel process for the preparation cis-nucleoside derivative. The present invention also relates to novel intermediates in the preparation of cis-nucleoside derivative. The present invention further relates to a pharmaceutical composition comprising cis-nucleoside derivative of formula-1 with excipients.

### BACKGROUND OF THE INVENTION

Cis-Nucleosides derivatives (formula-1, Lamivudine (R═H) and Emtricitabine (R═F)), are useful in the treatment of retroviral infections caused by Human immuno deficiency virus (HIV), Hepatitis B virus (HBV) and Human T-Lymotropic virus (HTLV).

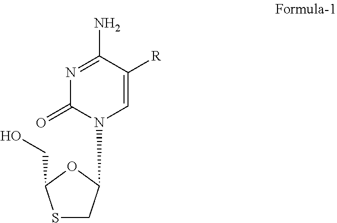

Lamivudine (3TC) is presently marketed by GlaxoSmithkline, is available as “EPIVIR”, and is disclosed first in U.S. Pat. No. 5,047,407. Emtricitabine is developed by Emory University, marketed by Gilead Sciences Inc., in the name of EMTRIVA and TRUVUDA, and is first disclosed in U.S. Pat. No. 5,814,639.

U.S. Pat. No. 5,047,407 describes the preparation of Lamivudine (3TC), its antiviral activity and its use in pharmaceutical product. According to US '407, Lamivudine is synthesized as a cis racemic mixture by reacting 5-ethoxy-2-benzyloxymethyl-[1,3]-oxathiolane with silylated cytosine followed by debenzoylation.

U.S. Pat. No. 5,814,639 describes Emtricitabine specifically and a pharmaceutical composition comprising an effective HIV treatment amount for humans of β-2′-deoxy-5-fluoro-3′-thiacytidine in a pharmaceutically acceptable carrier or diluent. More particularly, the invention relates to the β-isomers of these compounds and their selective synthesis and use as antiviral agents.

U.S. Pat. No. 5,696,254 describes a process to make compound of formula-1 by reacting 5-acetoxy-[1,3]-oxathiolane-2S-carboxylic acid-2S-isopropyl methyl-5R-methyl-1R-cyclohexyl ester with silylated cytosine derivative in the presence of silylated Lewis acid followed by reduction. However, the processes described in the above patents involve column chromatography.

U.S. Pat. No. 6,051,709 describes the process for the preparation of Lamivudine by reacting 5-chloro-[1,3]-oxathiolane-2S-carboxylic acid-2S-isopropylmethyl-5R-methyl-1R-cyclohexyl ester with silylated cytosine without Lewis acid, reduction of the obtained ester, followed by addition of salicylic acid to isolate Lamivudine as a salicylate salt. Further, the obtained salt is converted to Lamivudine. This process is restricted to specific leaving groups.

WO 2009069011A1 application describes a process for the preparation of Lamivudine using non-silylated Lewis acid during the condensation of (2S,5R)-2-isopropyl-5-methylcyclohexyl (2R)-5-(acetyloxy)-[1,3]-oxathiolane-2-carboxylate with N-acetyl silylated cytosine in the dichloroethane medium. This process involves non-silylated Lewis acid like SnCl4 and TiCl4, in which the work up is tedious.

We have developed novel intermediates and improved cost effective synthetic method to prepare cis-nucleoside derivative of formula-1 in industrial scale.

### SUMMARY OF THE INVENTION

The present invention relates to a novel process for the preparation cis-nucleoside derivative. The present invention also relates to novel intermediates in the preparation of cis-nucleoside derivative. The present invention further relates to a pharmaceutical composition comprising cis-nucleoside derivative of formula-1 with excipients.

One aspect of the present invention is to provide a process for the preparation of cis-nucleoside derivative of formula-1 comprising the steps of:

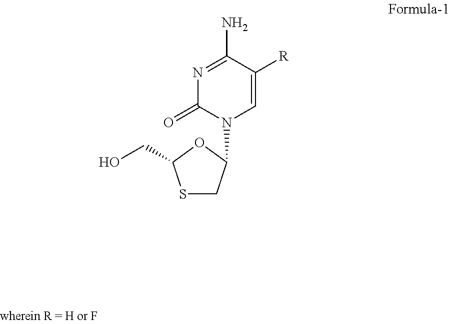

a) reacting the compound of formula-2 with chlorinating agent followed by compound of formula-3 in presence of a base to get compound of formula-4,

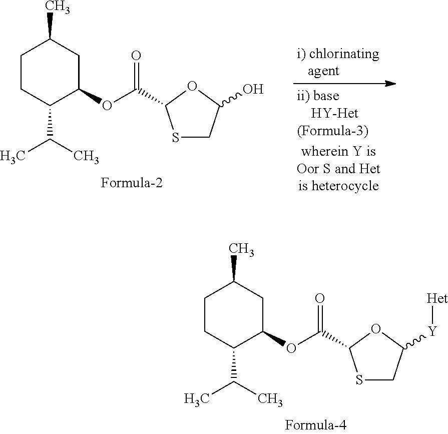

b) reacting the compound of formula-4 with an alkyl halide (R₁X) to get a quaternary ammonium salt then with cytosine derivative of formula-5 to provide the compound of formula-6,

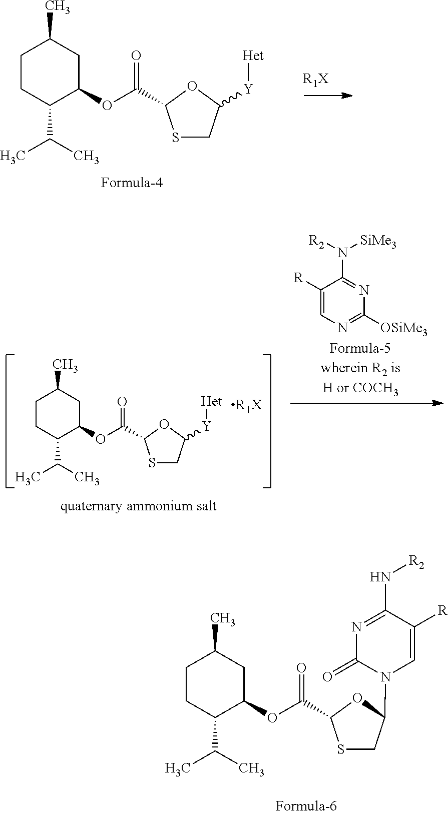

c) optionally de-protecting the compound of formula-6 to the compound of formula-7,

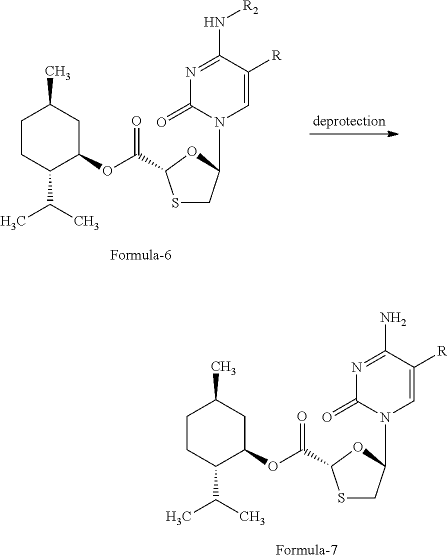

d) reducing compound of formula-7 with metal catalyst in presence of a buffer solution, then adding an organic acid to get the compound of formula-8, and

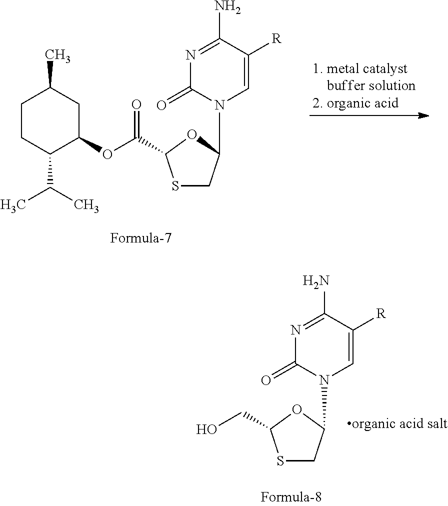

e) Converting the compound of formula-8 to cis-nucleoside derivative of formula-1.

Another aspect of the present invention is to provide a process for the preparation of Lamivudine comprising the steps of:

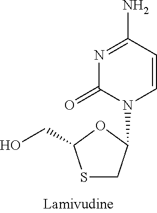

a) reacting the compound of formula-2 with chlorinating agent followed by compound of formula-3 in presence of a base to get compound of formula-4,

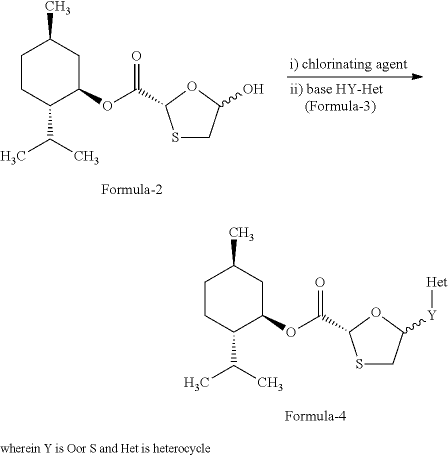

b) reacting the compound of formula-4 with an alkyl halide (R₁X) to get a quaternary ammonium salt and then with cytosine derivative of formula-5a to provide the compound of formula-6a,

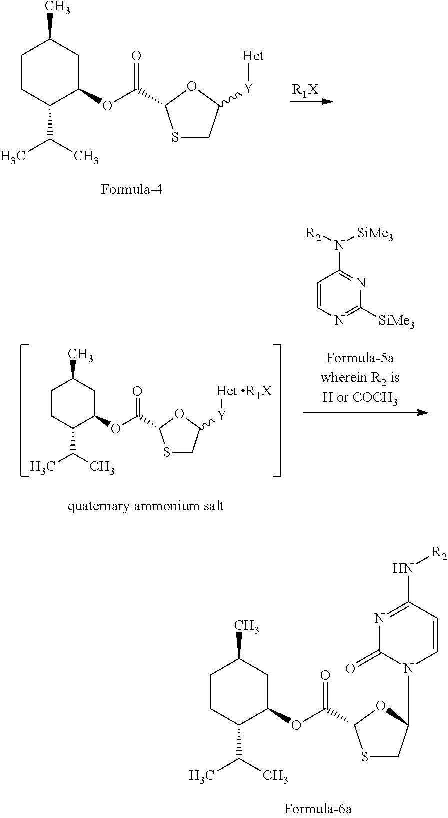

c) optionally de-protecting the compound of formula-6a to the compound of formula-7a,

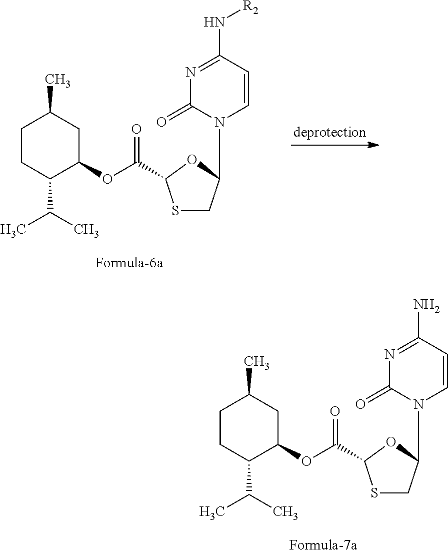

d) reducing compound of formula-7a with metal catalyst in presence of a buffer solution, then adding an organic acid to get the compound of formula-8a, and

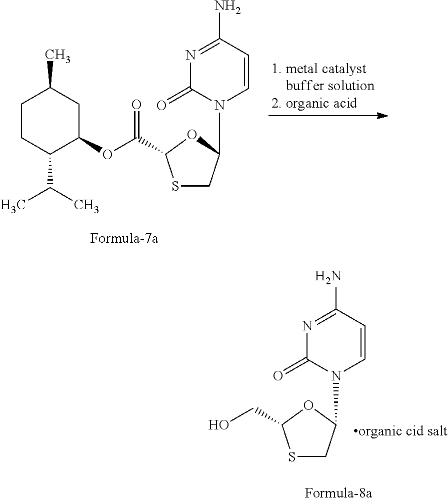

e) converting the compound of formula-8a to Lamivudine.

Yet another aspect of the present invention is to provide a process for the preparation of Emtricitabine comprising the steps of:

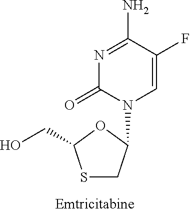

a) reacting the compound of formula-2 with chlorinating agent followed by compound of formula-3 in presence of a base to get compound of formula-4,

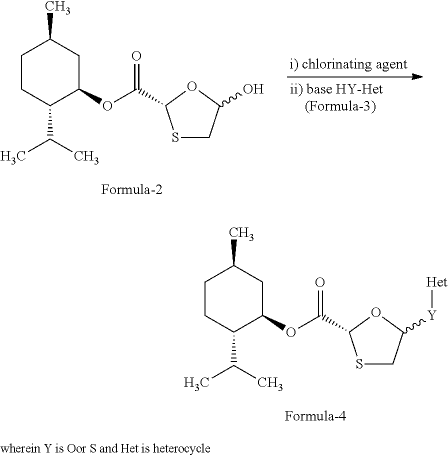

b) reacting the compound of formula-4 with an alkyl halide (R₁X) to get a quaternary ammonium salt and then with cytosine derivative of formula-5b to provide the compound of formula-6b,

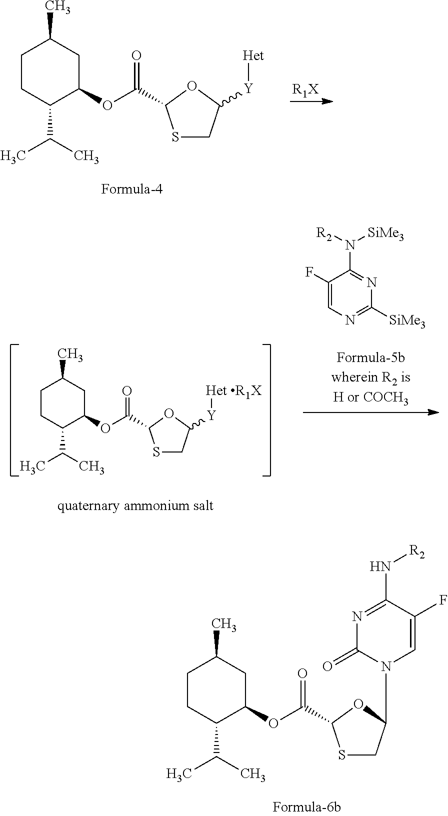

c) optionally de-protecting the compound of formula-6b to the compound of formula-7b,

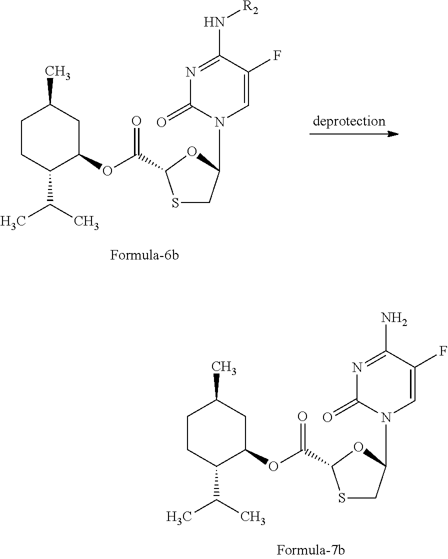

d) reducing compound of formula-7b with metal catalyst in presence of a buffer solution, then adding an organic acid to get the compound of formula-8b, and

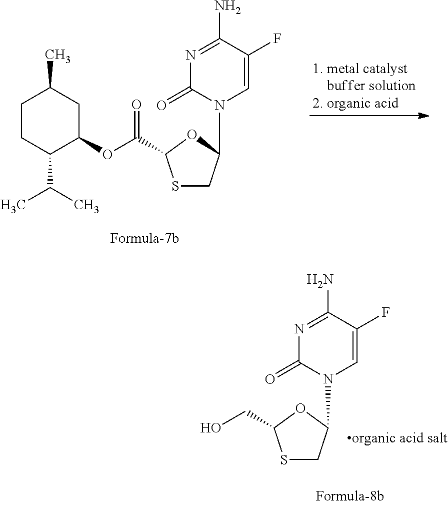

e) converting the compound of formula-8b to Emtricitabine.

Yet another aspect of the present invention is to provide a process for the preparation of compound of formula-4 comprising the steps of:
a. reacting the compound of formula-2 with chlorinating agent followed by condensing with compound of formula-3 in presence of a base, and
b. isolating the compound of formula-4,

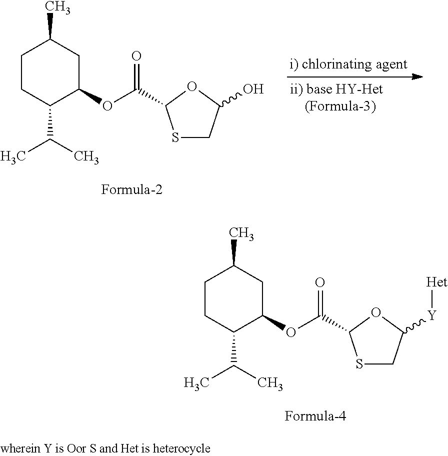

Yet another aspect of the present invention is to provide a process for the preparation of compound of formula-1 comprising the steps of:
a. dissolving the compound of formula-8 in an organic solvent,
b. treating with a base, and
c. isolating the compound of formula-1.

Yet another aspect of the present invention is to provide a process for the preparation of cis-nucleoside derivative of formula-8 from the compound of formula-4 comprising the steps of:
a) reacting the compound of formula-4 with an alkyl halide (RiX) to get a quaternary ammonium salt then with cytosine derivative of formula-5 to provide the compound of formula-6,

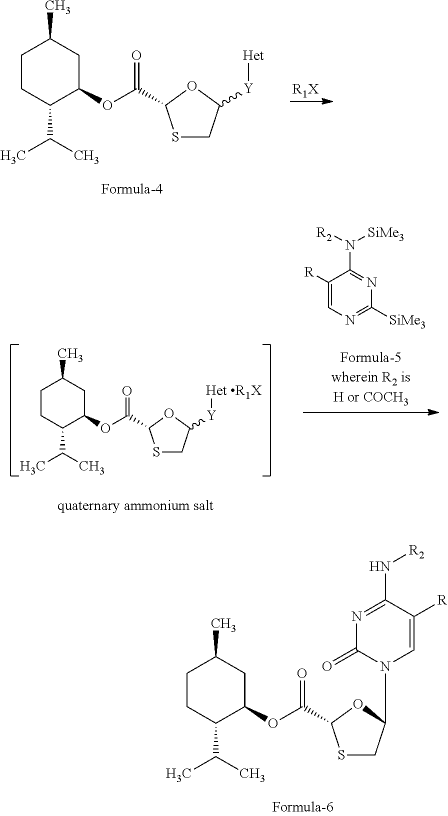

b) optionally de-protecting the compound of formula-6 to the compound of formula-7,

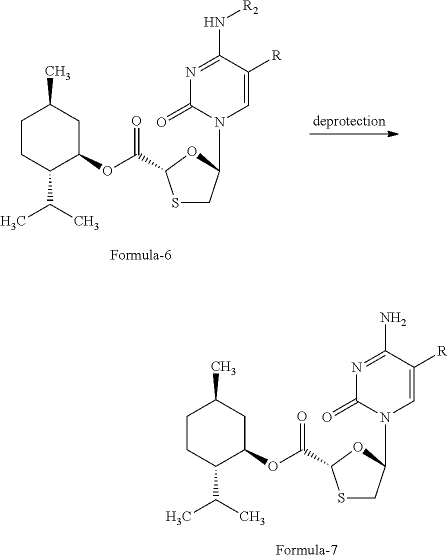

c) reducing compound of formula-7 with metal catalyst in presence of a buffer solution, then adding an organic acid to get the compound of formula-8.

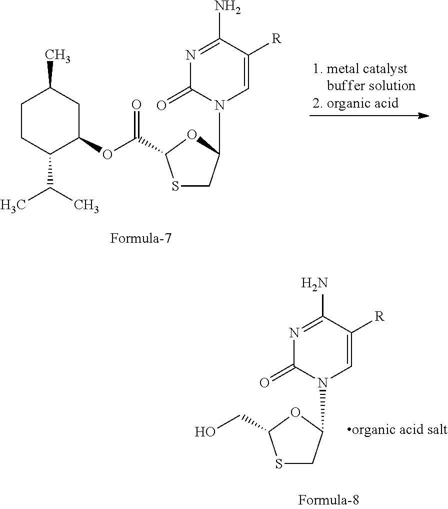

Yet another aspect of the present invention is to provide a novel compound of formula-4.

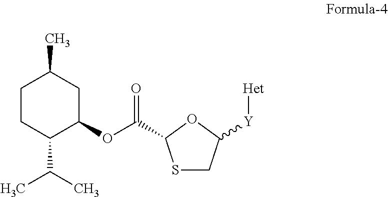

Yet another aspect of the present invention is to provide a novel compound of formula-4a.

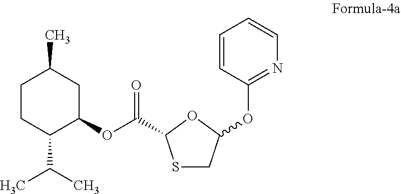

Yet another aspect of the present invention is to provide a novel compound of formula-4b.

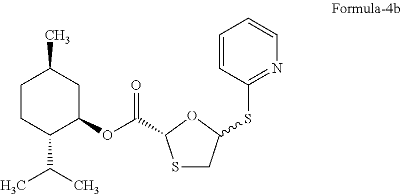

Yet another aspect of the present invention is to provide a novel compound of formula-8a1.

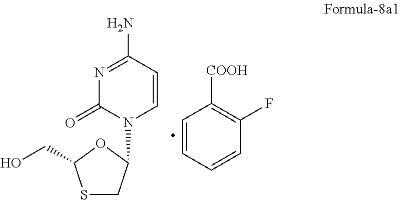

Yet another aspect of the present invention is to provide a novel compound of formula-8b1.

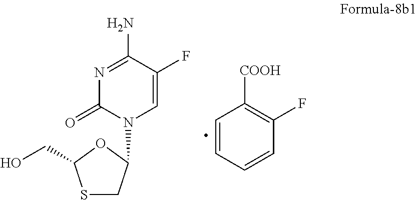

Yet another aspect of the present invention is to provide a novel compound of formula-8b2.

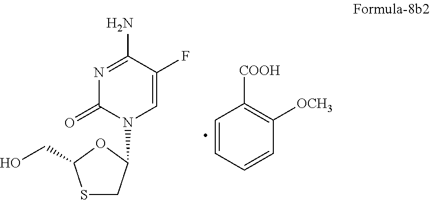

Yet another aspect of the present invention is to provide a novel compound of formula-8b3.

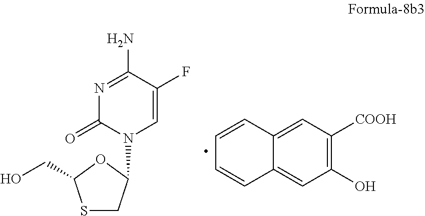

Yet another aspect of the present invention is to provide a novel compound of formula-8b4.

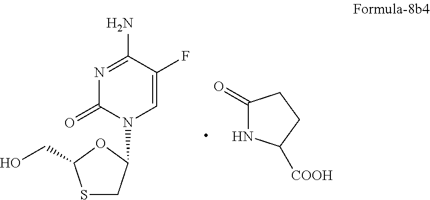

Yet another aspect of the present invention is to provide cis-nucleoside derivative of formula-1 compositions using (a) a therapeutically effective amount cis-nucleoside derivative of formula-1 or pharmaceutically acceptable salt; and (b) at least one pharmaceutically acceptable carrier.

The entire process for the preparation of cis-nucleoside derivative according to the present invention is as depicted in scheme 1 below.

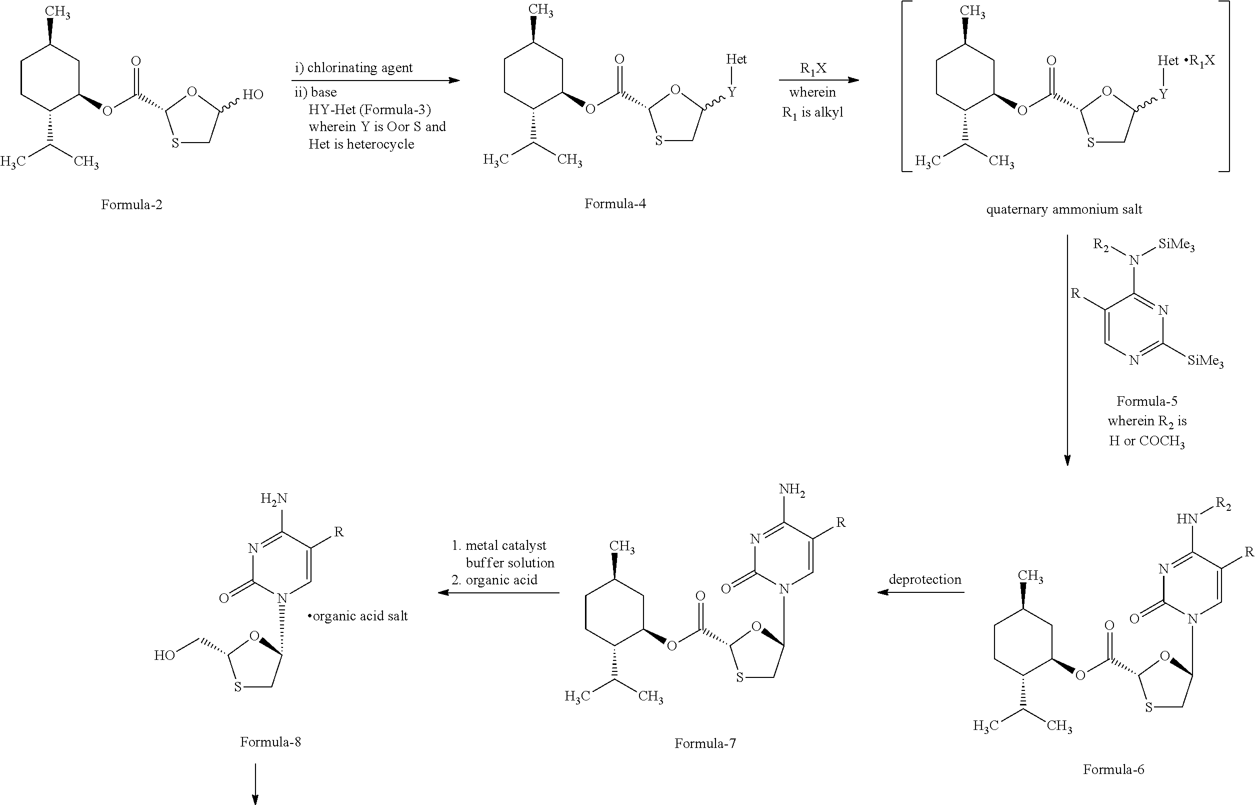

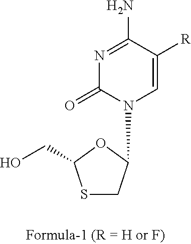

### DEFINITIONS

The term “heterocycle” represents a saturated or unsaturated mono- or polycyclic (i.e. bicyclic) ring incorporating 1 or more (i.e. 1-4) heteroatoms selected from N, O and S. It is understood that a heterocycle is optionally mono- or di-substituted with OH, SH, amino, halogen, CF₃, oxo or C₁₋₆ alkyl. Examples of suitable monocyclic heterocycles include but are not limited to pyridine, piperidine, pyrazine, piperazine, pyrimidine, imidazole, thiazole, oxazole, furan, pyran and thiophene. Examples of suitable bicyclic heterocycles include but are not limited to indole, benzimidazole, benzothiazole quinoline, isoquinoline, purine, and carbazole.

The term “aryl” refers to aromatic homocyclic (i.e., hydrocarbon) mono-, bi- or tricyclic ring-containing groups such as having 6 to 12 members such as phenyl, naphthyl and biphenyl. The term “aryl” also refers to phenyl (optionally substituted).

The terms “halogen” and “halo” refer to fluorine, chlorine, bromine and iodine.

The term “suspension”, “suspended” or “suspend” represents soluble, insoluble or partially soluble.

### DETAILED DESCRIPTION OF THE INVENTION

The present invention relates to an improved process for the preparation of cis-nucleoside derivative of formula-1 involving chlorination of the compound of formula-2 followed by reaction with compound of formula-3 in presence of a base to get compound of formula-4, reacting the compound of formula-4 with an alkyl halide (R₁X) to get a quaternary ammonium salt then with cytosine derivative of formula-5 to provide the compound of formula-6, optionally de-protecting the compound of formula-6 to the compound of formula-7, reducing compound of formula-7 with metal catalyst in presence of a buffer solution, then adding an organic acid to get the compound of formula-8, and converting the compound of formula-8 to cis-nucleoside derivative of formula-1. The present invention further relates to novel cis-nucleoside derivative of formula-8. The present invention also relates to a pharmaceutical composition comprising cis-nucleoside derivative of formula-1 with excipients.

One embodiment of the present invention is to provide a process for the preparation of cis-nucleoside derivative of formula-1 comprising the steps of:

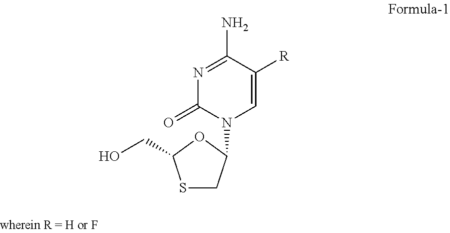

a) reacting the compound of formula-2 with chlorinating agent followed by compound of formula-3 in presence of a base to get compound of formula-4,

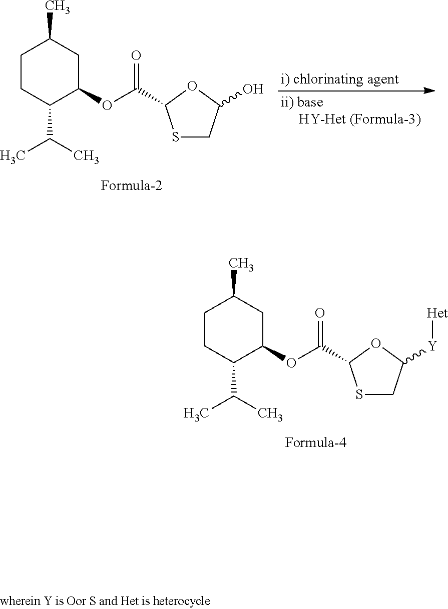

b) reacting the compound of formula-4 with an alkyl halide (R₁X) to get a quaternary ammonium salt then with cytosine derivative of formula-5 to provide the compound of formula-6,

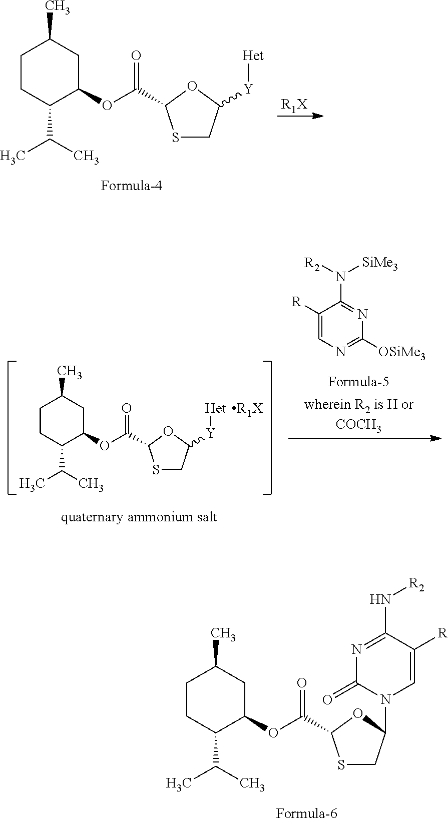

c) optionally de-protecting the compound of formula-6 to the compound of formula-7,

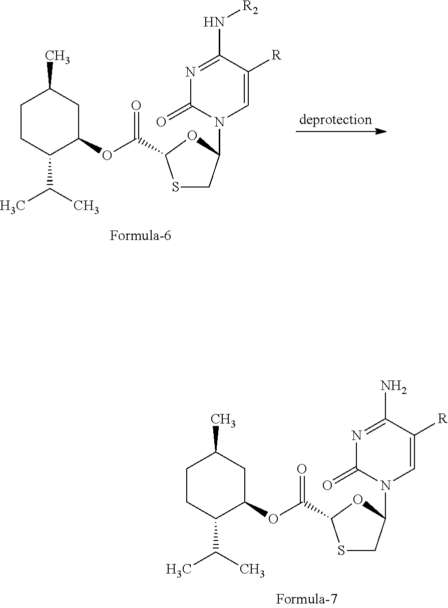

d) reducing compound of formula-7 with metal catalyst in presence of a buffer solution, then adding an organic acid to get the compound of formula-8, and

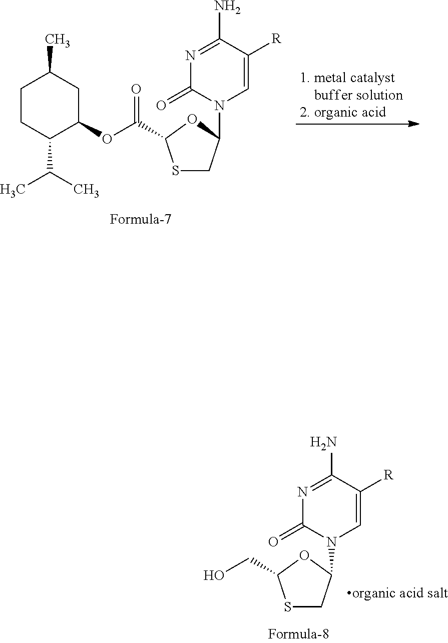

e) Converting the compound of formula-8 to cis-nucleoside derivative of formula-1.

According to the present invention, the compound of formula-2 is dissolved in a solvent optionally containing catalytic amount of N,N-dimethylformamide and methanesulphonic acid. The mixture is reacted with chlorinating agent at 10-25° C. to form corresponding chloro compound, then condensing with compound of formula-3 in presence of a base to get the compound of formula-4.

The solvent is selected from dichloromethane, chloroform, dichloroethane, acetone, tetrahydrofuran, dimethylformamide, dimethyl sulphoxide or mixture thereof.

The chlorinating agent is selected from phosphorus pentachloride, phosphorus trichloride, thionyl chloride or triphenylphosphine dichloride.

The compound of formula-3 is selected from 2-mercaptopyridine, 4-mercaptopyridine, 2-hydroxypyridine, 4-hydroxypyridine, alkyl-2-mercaptopyridine, alkyl-4-mercaptopyridine, alkyl-2-hydroxypyridine, alkyl-4-hydroxypyridine, heteryl-2-mercaptopyridine, heteryl-4-mercaptopyridine, heteryl-2-hydroxypyridine, heteryl-4-hydroxypyridine, alkoxy-2-mercaptopyridine, alkoxy-4-mercaptopyridine, aryloxy-2-mercaptopyridine, aryloxy-4-mercaptopyridine, alkoxy-2-hydroxypyridine, alkoxy-4-hydroxypyridine, aryloxy-2-hydroxypyridine, aryloxy-4-hydroxypyridine, alkyloxycarbonyl-2-mercaptopyridine, alkyloxycarbonyl-4-mercaptopyridine, aryloxycarbonyl-2-mercaptopyridine, aryloxy-carbonyl-4-mercaptopyridine, alkyloxycarbonyl-2-hydroxypyridine, alkyloxycarbonyl-4-hydroxypyridine, aryloxycarbonyl-2-hydroxypyridine, aryloxycarbonyl-4-hydroxypyridine, 1,3-benzothiazol-2-ol, alkyl-1,3-benzothiazol-2-ol, alkoxy-1,3-benzothiazol-2-ol, 1,3-benzothiazol-2-thiol, alkyl-1,3-benzothiazol-2-thiol or alkoxy-1,3-benzothiazol-2-thiol.

The base used for the condensation is selected from organic bases such as triethylamine, tributylamine, N-methylmorpholine, N,N-diisopropylethylamine, N-methylpyrrolidine, pyridine; 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

According to the present invention, compound of formula-4 is reacted with alkyl halide in a solvent or mixture thereof to get quaternary ammonium salt, which is in-situ reacted with cytosine derivative of formula-5 optionally in the presence of molecular sieves at 50-100° C. to get compound of formula-6.

The compound of formula-4 is dissolved in a solvent selected from toluene, acetone, dichloromethane, chloroform, dichloroethane, tetrahydrofuran, dimethylformamide, dimethyl sulfoxide or mixture thereof.

The alkyl halide used in the reaction is selected from methyl iodide, methyl bromide, ethyl iodide, ethyl bromide, butyl iodide, butyl bromide, trityl chloride, p-toluenesulphonyl chloride or methyl triflate.

According to the present invention, the compound of formula-6 is treated with an acid in a solvent at 10-40° C. for the deprotection and isolating the corresponding acid salt of formula-7. The obtained acid salt of formula-7 is reacted with a base in a solvent at 10-40° C. to get the compound of formula-7. However, the compound of formula-7 is also isolated in single step by treating the compound of formula-6 with an acid in a solvent at 10-40° C. followed by a base to adjust the pH of the reaction mass.

The solvents used for the dissolution of the compound of formula-6 or acid salt of formula-7 is selected from methanol, ethanol, isopropyl alcohol, n-butanol, iso-butanol, acetone, methyl isobutyl ketone, hexane, heptane, octane, ethyl acetate, propyl acetate, methyl acetate, tetrahydrofuran, dioxane, chloroform, dichloromethane, water or mixture thereof.

The acid used for the de-protection of the compound of formula-6 is selected from hydrochloric acid, sulfuric acid, methansulfonic acid, phosphoric acid, formic acid, acetic acid, benzenesulfonic acid or p-toluenesulfonic acid.

The base used for the reaction to adjust the pH of the reaction is selected from organic base such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

According to the present invention, compound of formula-7 is dissolved in a solvent or mixture thereof and reduced with metal catalyst at 15-45° C. in presence of a buffer solution to get crude cis-nucleoside derivative of formula-1. The crude cis-nucleoside derivative of formula-1 is treated with an organic acid at ambient temperature for 2-8 h to isolate cis-nucleoside derivative of formula-8, which is having less solubility as compared to the prior art acid salts. Hence, cis-nucleoside derivative of formula-8 is isolated with improved yield and quality.

The solvent used for the dissolution of compound of formula-7 is selected from ethanol, methanol, n-propanol, 2-propanol, N,N-dimethylformamide, tetrahydrofuran, water or mixture thereof.

The metal catalyst used for the reduction of the compound of formula-7 is selected from sodium borohydride, potassium borohydride, lithium borohydride or lithium aluminium hydride.

The buffer solution used in the reduction is selected from disodium hydrogen phosphate or dipotassium hydrogen orthophosphate.

The organic acid is selected from aromatic acids such as halobenzoic acids like 2-fluorobenzoic acid, 3-fluorobenzoic acid, 4-fluorobenzoic acid, 2-chlorobenzoic acid, 3-chlorobenzoic acid, 4-chlorobenzoic acid, 2-bromobenzoic acid, 3-bromobenzoic acid, 4-bromobenzoic acid, 2-iodobenzoic acid, 3-iodobenzoic acid or 4-iodobenzoic acid. Other organic acids includes 3-hydroxy-2-naphthoic acid, 2-methoxybenzoic acid, 3-methoxybenzoic acid, 4-methoxybenzoic acid, 2-methylbenzoic acid, 3-methylbenzoic acid, 4-methylbenzoic acid, 3-methylsalicylic acid, 4-methylsalicylic acid, 5-bromosalicylic acid, 3-methoxysalicylic acid, 4-methoxysalicylic acid or amino acids such as L-pyroglutamic acid or aspartic acid.

According to the present invention, the suspension of cis-nucleoside derivative of formula-8 is treated with a base in a solvent or mixture of solvent and is isolated cis-nucleoside derivative of formula-1.

The solvent used to suspend the compound of formula-8 is selected from methanol, ethanol, isopropyl alcohol, n-butanol, iso-butanol, acetone, ethyl acetate, isopropyl acetate, tetrahydrofuran, dioxane, water or mixture thereof. The base used is selected from triethylamine, Hunig's base or ammonia.

Another embodiment of the present invention is to provide a process for the preparation of Lamivudine comprising the steps of:

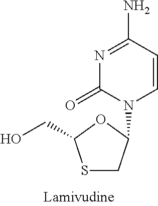

a) reacting the compound of formula-2 with chlorinating agent followed by compound of formula-3 in presence of a base to get compound of formula-4,

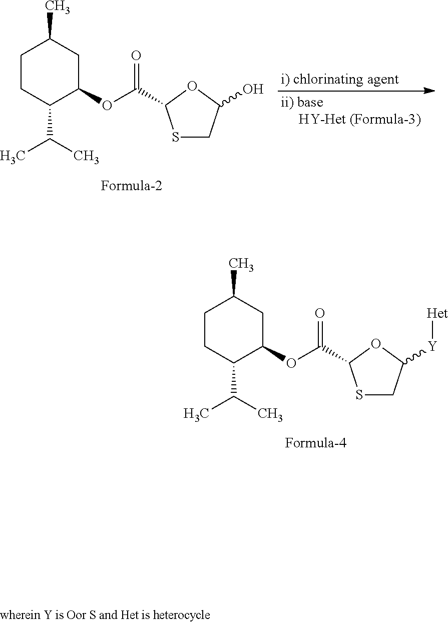

b) reacting the compound of formula-4 with an alkyl halide (R₁X) to get a quaternary ammonium salt and then with cytosine derivative of formula-5a to provide the compound of formula-6a,

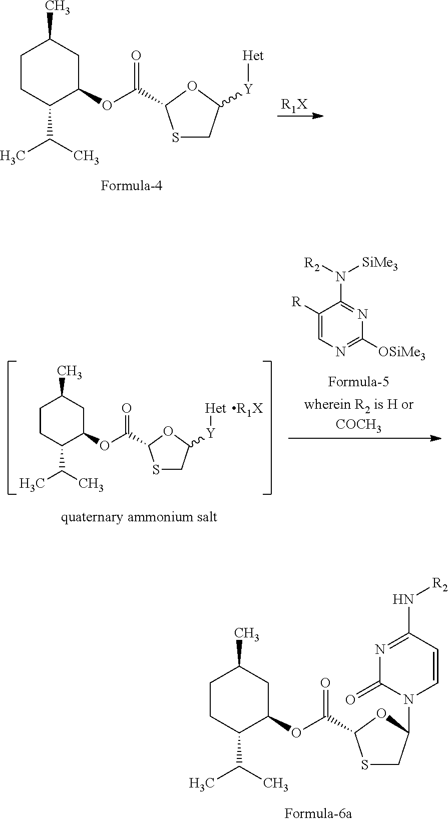

c) optionally de-protecting the compound of formula-6a to the compound of formula-7a,

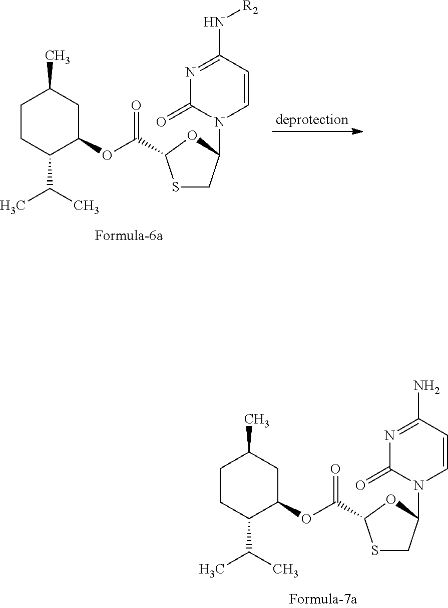

d) reducing compound of formula-7a with metal catalyst in presence of a buffer solution, then adding an organic acid to get the compound of formula-8a, and

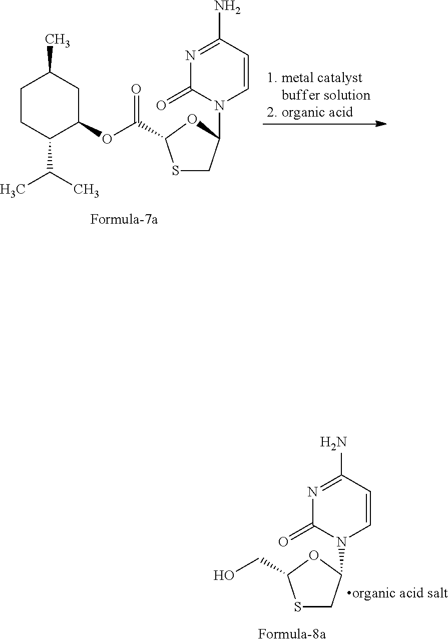

e) converting the compound of formula-8a to Lamivudine.

According to the present invention, the compound of formula-2 is dissolved in a solvent optionally containing catalytic amount of N,N-dimethylformamide and methanesulphonic acid. The mixture is reacted with chlorinating agent at 10-25° C. to form corresponding chloro compound, then condensing with compound of formula-3 in presence of a base to get the compound of formula-4.

The solvent is selected from dichloromethane, chloroform, dichloroethane, acetone, tetrahydrofuran, dimethylformamide, dimethyl sulphoxide or mixture thereof.

The chlorinating agent is selected from phosphorus pentachloride, phosphorus trichloride, thionyl chloride or triphenylphosphine dichloride.

The compound of formula-3 is selected from 2-mercaptopyridine, 4-mercaptopyridine, 2-hydroxypyridine, 4-hydroxypyridine, alkyl-2-mercaptopyridine, alkyl-4-mercaptopyridine, alkyl-2-hydroxypyridine, alkyl-4-hydroxypyridine, heteryl-2-mercaptopyridine, heteryl-4-mercaptopyridine, heteryl-2-hydroxypyridine, heteryl-4-hydroxypyridine, alkoxy-2-mercapto-pyridine, alkoxy-4-mercaptopyridine, aryloxy-2-mercaptopyridine, aryloxy-4-mercapto-pyridine, alkoxy-2-hydroxypyridine, alkoxy-4-hydroxypyridine, aryloxy-2-hydroxypyridine, aryloxy-4-hydroxypyridine, alkyloxycarbonyl-2-mercaptopyridine, alkyloxycarbonyl-4-mercaptopyridine, aryloxycarbonyl-2-mercaptopyridine, aryloxy-carbonyl-4-mercapto-pyridine, alkyloxycarbonyl-2-hydroxypyridine, alkyloxycarbonyl-4-hydroxypyridine, aryloxy-carbonyl-2-hydroxypyridine, aryloxycarbonyl-4-hydroxypyridine, 1,3-benzothiazol-2-ol, alkyl-1,3-benzothiazol-2-ol, alkoxy-1,3-benzothiazol-2-ol, 1,3-benzothiazol-2-thiol, alkyl-1,3-benzothiazol-2-thiol or alkoxy-1,3-benzothiazol-2-thiol.

The base used for the condensation is selected from organic bases such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

According to the present invention, compound of formula-4 is reacted with alkyl halide in a solvent or mixture thereof to get quaternary ammonium salt, which is in-situ reacted with cytosine derivative of formula-5a optionally in the presence of molecular sieves at 50-100° C. to get compound of formula-6a.

The compound of formula-4 is dissolved in a solvent selected from toluene, acetone, dichloromethane, chloroform, dichloroethane, tetrahydrofuran, dimethylformamide, dimethyl sulfoxide or mixture thereof.

The alkyl halide used in the reaction is selected from methyl iodide, methyl bromide, ethyl iodide, ethyl bromide, butyl iodide, butyl bromide, trityl chloride, p-toluenesulphonyl chloride or methyl triflate.

According to the present invention, the compound of formula-6a is treated with an acid in a solvent at 10-40° C. for the deprotection and isolating the corresponding acid salt of formula-7a. The obtained acid salt of formula-7a is reacted with a base in a solvent at 10-40° C. to get the compound of formula-7a. However, the compound of formula-7a is also isolated in single step by treating the compound of formula-6 with an acid in a solvent at 10-40° C. followed by a base to adjust the pH of the reaction mass.

The solvents used for the dissolution of the compound of formula-6a or acid salt of formula-7a is selected from methanol, ethanol, isopropyl alcohol, n-butanol, iso-butanol, acetone, methyl isobutyl ketone, hexane, heptane, octane, ethyl acetate, propyl acetate, methyl acetate, tetrahydrofuran, dioxane, chloroform, dichloromethane, water or mixture thereof.

The acid used for the de-protection of the compound of formula-6a is selected from hydrochloric acid, sulfuric acid, methansulfonic acid, phosphoric acid, formic acid, acetic acid, benzenesulfonic acid or p-toluenesulfonic acid.

The base used for the reaction to adjust the pH of the reaction is selected from organic base such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

According to the present invention, compound of formula-7a is dissolved in a solvent or mixture thereof and reduced with metal catalyst at 15-45° C. in presence of a buffer solution to get crude Lamivudine. The crude Lamivudine is treated with an organic acid at ambient temperature for 2-8 h to isolate cis-nucleoside derivative of formula-8a, which is having less solubility as compared to the prior art acid salts. Hence, cis-nucleoside derivative of formula-8a is isolated with improved yield and quality.

The solvent used for the dissolution of compound of formula-7a is selected from ethanol, methanol, n-propanol, 2-propanol, N,N-dimethylformamide, tetrahydrofuran, water or mixture thereof.

The metal catalyst used for the reduction of the compound of formula-7a is selected from sodium borohydride, potassium borohydride, lithium borohydride or lithium aluminium hydride.

The buffer solution used in the reduction is selected from disodium hydrogen phosphate or dipotassium hydrogen orthophosphate.

The organic acid is selected from aromatic acids such as halobenzoic acids like 2-fluorobenzoic acid, 3-fluorobenzoic acid, 4-fluorobenzoic acid, 2-chlorobenzoic acid, 3-chlorobenzoic acid, 4-chlorobenzoic acid, 2-bromobenzoic acid, 3-bromobenzoic acid, 4-bromobenzoic acid, 2-iodobenzoic acid, 3-iodobenzoic acid or 4-iodobenzoic acid. Other organic acids includes 3-hydroxy-2-naphthoic acid, 2-methoxybenzoic acid, 3-methoxybenzoic acid, 4-methoxybenzoic acid, 2-methylbenzoic acid, 3-methylbenzoic acid, 4-methylbenzoic acid, 3-methylsalicylic acid, 4-methylsalicylic acid, 5-bromosalicylic acid, 3-methoxysalicylic acid, 4-methoxysalicylic acid or amino acids such as L-pyroglutamic acid or aspartic acid.

According to the present invention, the suspension of cis-nucleoside derivative of formula-8a is treated with a base in a solvent or mixture of solvent and is isolated Lamivudine.

The solvent used to suspend the compound of formula-8a is selected from methanol, ethanol, isopropyl alcohol, n-butanol, iso-butanol, acetone, ethyl acetate, isopropyl acetate, tetrahydrofuran, dioxane, water or mixture thereof. The base used is selected from triethylamine, Hunig's base or ammonia.

Yet another embodiment of the present invention is to provide a process for the preparation of Emtricitabine comprising the steps of:

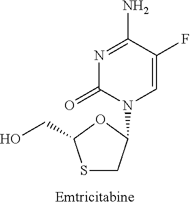

a) reacting the compound of formula-2 with chlorinating agent followed by compound of formula-3 in presence of a base to get compound of formula-4,

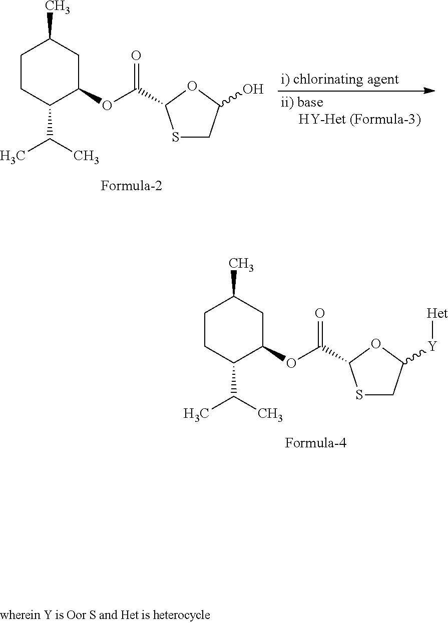

b) reacting the compound of formula-4 with an alkyl halide (R₁X) to get a quaternary ammonium salt and then with cytosine derivative of formula-5b to provide the compound of formula-6b,

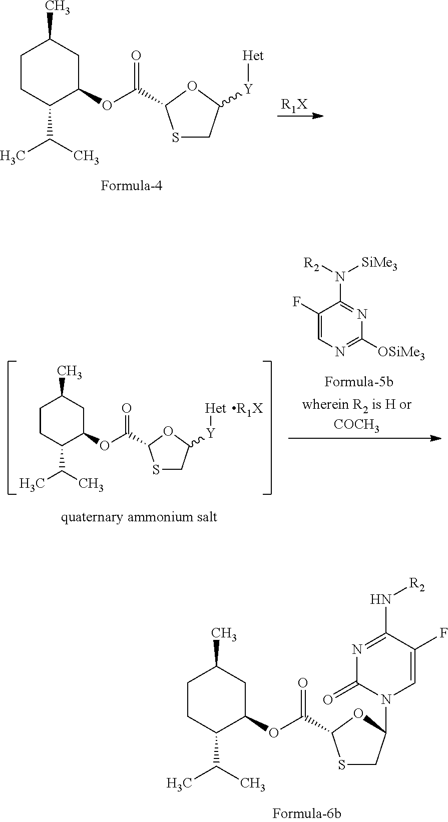

c) optionally de-protecting the compound of formula-6b to the compound of formula-7b,

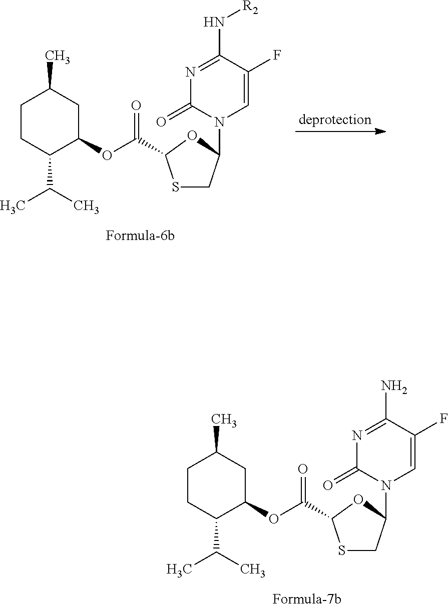

d) reducing compound of formula-7b with metal catalyst in presence of a buffer solution, then adding an organic acid to get the compound of formula-8b, and

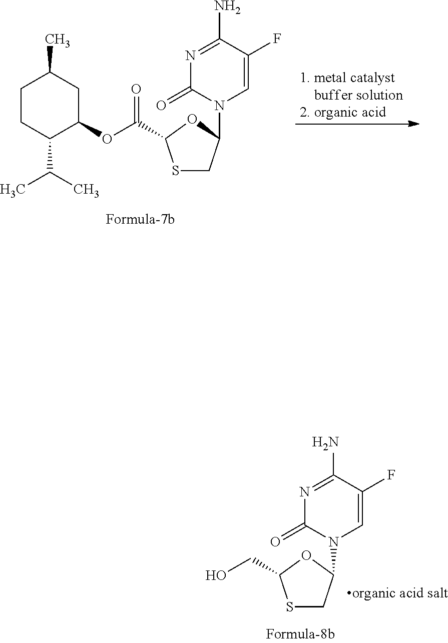

e) converting the compound of formula-8b to Emtricitabine.

According to the present invention, the compound of formula-2 is dissolved in a solvent optionally containing catalytic amount of N,N-dimethylformamide and methanesulphonic acid. The mixture is reacted with chlorinating agent at 10-25° C. to form corresponding chloro compound, then condensing with compound of formula-3 in presence of a base to get the compound of formula-4.

The solvent is selected from dichloromethane, chloroform, dichloroethane, acetone, tetrahydrofuran, dimethylformamide, dimethyl sulphoxide or mixture thereof.

The chlorinating agent is selected from phosphorus pentachloride, phosphorus trichloride, thionyl chloride or triphenylphosphine dichloride.

The compound of formula-3 is selected from 2-mercaptopyridine, 4-mercaptopyridine, 2-hydroxypyridine, 4-hydroxypyridine, alkyl-2-mercaptopyridine, alkyl-4-mercaptopyridine, alkyl-2-hydroxypyridine, alkyl-4-hydroxypyridine, heteryl-2-mercaptopyridine, heteryl-4-mercaptopyridine, heteryl-2-hydroxypyridine, heteryl-4-hydroxypyridine, alkoxy-2-mercapto-pyridine, alkoxy-4-mercaptopyridine, aryloxy-2-mercaptopyridine, aryloxy-4-mercapto-pyridine, alkoxy-2-hydroxypyridine, alkoxy-4-hydroxypyridine, aryloxy-2-hydroxypyridine, aryloxy-4-hydroxypyridine, alkyloxycarbonyl-2-mercaptopyridine, alkyloxycarbonyl-4-mercaptopyridine, aryloxycarbonyl-2-mercaptopyridine, aryloxy-carbonyl-4-mercapto-pyridine, alkyloxycarbonyl-2-hydroxypyridine, alkyloxycarbonyl-4-hydroxypyridine, aryloxy-carbonyl-2-hydroxypyridine, aryloxycarbonyl-4-hydroxypyridine, 1,3-benzothiazol-2-ol, alkyl-1,3-benzothiazol-2-ol, alkoxy-1,3-benzothiazol-2-ol, 1,3-benzothiazol-2-thiol, alkyl-1,3-benzothiazol-2-thiol or alkoxy-1,3-benzothiazol-2-thiol.

The base used for the condensation is selected from organic bases such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

According to the present invention, compound of formula-4 is reacted with alkyl halide in a solvent or mixture thereof to get quaternary ammonium salt, which is in-situ reacted with cytosine derivative of formula-5b optionally in the presence of molecular sieves at 50-100° C. to get compound of formula-6b.

The compound of formula-4 is dissolved in a solvent selected from toluene, acetone, dichloromethane, chloroform, dichloroethane, tetrahydrofuran, dimethylformamide, dimethyl sulfoxide or mixture thereof.

The alkyl halide used in the reaction is selected from methyl iodide, methyl bromide, ethyl iodide, ethyl bromide, butyl iodide, butyl bromide, trityl chloride, p-toluenesulphonyl chloride or methyl triflate.

According to the present invention, the compound of formula-6b is treated with an acid in a solvent at 10-40° C. for the deprotection and isolating the corresponding acid salt of formula-7b. The obtained acid salt of formula-7b is reacted with a base in a solvent at 10-40° C. to get the compound of formula-7b. However, the compound of formula-7b is also isolated in single step by treating the compound of formula-6b with an acid in a solvent at 10-40° C. followed by a base to adjust the pH of the reaction mass.

The solvents used for the dissolution of the compound of formula-6b or acid salt of formula-7b is selected from methanol, ethanol, isopropyl alcohol, n-butanol, iso-butanol, acetone, methyl isobutyl ketone, hexane, heptane, octane, ethyl acetate, propyl acetate, methyl acetate, tetrahydrofuran, dioxane, chloroform, dichloromethane, water or mixture thereof.

The acid used for the de-protection of the compound of formula-6b is selected from hydrochloric acid, sulfuric acid, methansulfonic acid, phosphoric acid, formic acid, acetic acid, benzenesulfonic acid or p-toluenesulfonic acid.

The base used for the reaction to adjust the pH of the reaction is selected from organic base such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium, and the like; and other suitable bases.

According to the present invention, compound of formula-7b is dissolved in a solvent or mixture thereof and reduced with metal catalyst at 15-45° C. in presence of a buffer solution to get crude Emtricitabine. The crude Emtricitabine is treated with an organic acid at ambient temperature for 2-8 h to isolate cis-nucleoside derivative of formula-8b, which is having less solubility as compared to the prior art acid salts. Hence, cis-nucleoside derivative of formula-8b is isolated with improved yield and quality.

The solvent used for the dissolution of compound of formula-7b is selected from ethanol, methanol, n-propanol, 2-propanol, N,N-dimethylformamide, tetrahydrofuran, water or mixture thereof.

The metal catalyst used for the reduction of the compound of formula-7b is selected from sodium borohydride, potassium borohydride, lithium borohydride or lithium aluminium hydride.

The buffer solution used in the reduction is selected from disodium hydrogen phosphate or dipotassium hydrogen orthophosphate.

The organic acid is selected from aromatic acids such as halobenzoic acids like 2-fluorobenzoic acid, 3-fluorobenzoic acid, 4-fluorobenzoic acid, 2-chlorobenzoic acid, 3-chlorobenzoic acid, 4-chlorobenzoic acid, 2-bromobenzoic acid, 3-bromobenzoic acid, 4-bromobenzoic acid, 2-iodobenzoic acid, 3-iodobenzoic acid or 4-iodobenzoic acid. Other organic acids includes 3-hydroxy-2-naphthoic acid, 2-methoxybenzoic acid, 3-methoxybenzoic acid, 4-methoxybenzoic acid, 2-methylbenzoic acid, 3-methylbenzoic acid, 4-methylbenzoic acid, 3-methylsalicylic acid, 4-methylsalicylic acid, 5-bromosalicylic acid, 3-methoxysalicylic acid, 4-methoxysalicylic acid or amino acids such as L-pyroglutamic acid or aspartic acid.

According to the present invention, the suspension of cis-nucleoside derivative of formula-8b is treated with a base in a solvent or mixture of solvent and is isolated Emtricitabine.

The solvent used to suspend the compound of formula-8b is selected from methanol, ethanol, isopropyl alcohol, n-butanol, iso-butanol, acetone, ethyl acetate, isopropyl acetate, tetrahydrofuran, dioxane, water or mixture thereof. The base used is selected from triethylamine, Hunig's base or ammonia.

Yet another embodiment of the present invention is to provide a process for the preparation of compound of formula-4 comprising the steps of:
a. reacting the compound of formula-2 with chlorinating agent followed by condensing with compound of formula-3 in presence of a base, and
b. isolating the compound of formula-4,

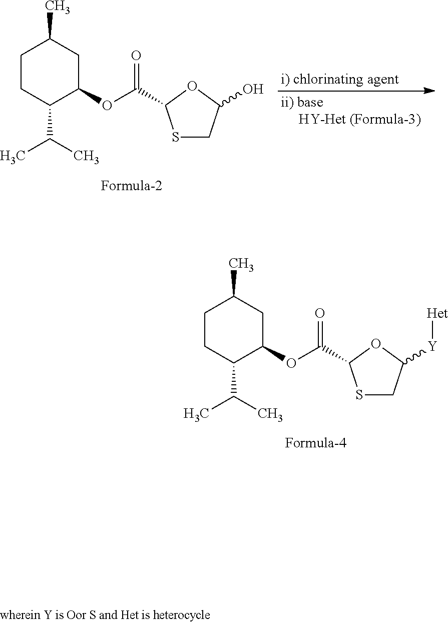

According to the present invention, the compound of formula-2 is dissolved in a solvent containing N,N-dimethylformamide and methanesulphonic acid and reacted with chlorinating agent at 10-25° C. to form corresponding chloro compound then condensing with compound of formula-3 in presence of a base to get the compound of formula-4.

The solvent is selected from dichloromethane, chloroform, dichloroethane, acetone, tetrahydrofuran, dimethylformamide, dimethyl sulphoxide or mixture thereof.

The chlorinating agent is selected from phosphorus pentachloride, phosphorus trichloride, thionyl chloride or triphenylphosphine dichloride.

The compound of formula-3 is selected from 2-mercaptopyridine, 4-mercaptopyridine, 2-hydroxypyridine, 4-hydroxypyridine, alkyl-2-mercaptopyridine, alkyl-4-mercaptopyridine, alkyl-2-hydroxypyridine, alkyl-4-hydroxypyridine, heteryl-2-mercaptopyridine, heteryl-4-mercaptopyridine, heteryl-2-hydroxypyridine, heteryl-4-hydroxypyridine, alkoxy-2-mercapto-pyridine, alkoxy-4-mercaptopyridine, aryloxy-2-mercaptopyridine, aryloxy-4-mercapto-pyridine, alkoxy-2-hydroxypyridine, alkoxy-4-hydroxypyridine, aryloxy-2-hydroxypyridine, aryloxy-4-hydroxypyridine, alkyloxycarbonyl-2-mercaptopyridine, alkyloxycarbonyl-4-mercaptopyridine, aryloxycarbonyl-2-mercaptopyridine, aryloxy-carbonyl-4-mercapto-pyridine, alkyloxycarbonyl-2-hydroxypyridine, alkyloxycarbonyl-4-hydroxypyridine, aryloxy-carbonyl-2-hydroxypyridine, aryloxycarbonyl-4-hydroxypyridine, 1,3-benzothiazol-2-ol, alkyl-1,3-benzothiazol-2-ol, alkoxy-1,3-benzothiazol-2-ol, 1,3-benzothiazol-2-thiol, alkyl-1,3-benzothiazol-2-thiol or alkoxy-1,3-benzothiazol-2-thiol.

The base used for the condensation is selected from organic bases such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

Yet another embodiment of the present invention is to provide a process for the preparation of compound of formula-4 by reacting compound of formula-2 with pyridine disulfide derivative of formula-9 in a organic solvent containing triarylphosphine.

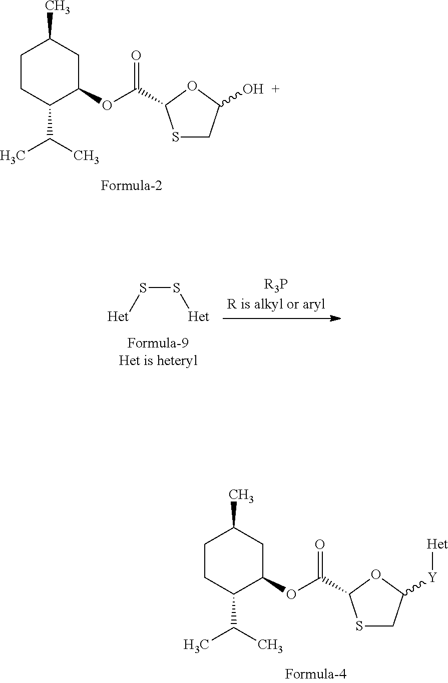

The solvent is selected from dichloromethane, chloroform, dichloroethane, acetone, tetrahydrofuran, toluene, N,N-dimethylformamide or mixture thereof.

The heteryl in compound formula-9 is selected from 2-pyridyl, 5-nitro-2-pyridyl or 4-pyridyl.

The triarylphosphine is selected from triphenylphosphine or tri(o-tolyl)phosphine.

Yet another embodiment of the present invention is to provide a process for the preparation of cis-nucleoside derivative of formula-1 comprising the steps of:
a. suspending the compound of formula-8 in an organic solvent,
b. treating with a base, and
c. isolating the cis-nucleoside derivative of formula-1.

According to the present invention, the suspension of the compound of formula-8 is treated with a base in a solvent or mixture of solvent and then isolating cis-nucleoside derivative of formula-1.

The solvent used to suspend the compound of formula-8 is selected from methanol, ethanol, isopropyl alcohol, n-butanol, iso-butanol, acetone, ethyl acetate, isopropyl acetate, tetrahydrofuran, dioxane, water or mixture thereof. The base used is selected from triethylamine, Hunig's base or ammonia.

Yet another embodiment of the present invention is to provide a process for the preparation of cis-nucleoside derivative of formula-8 from the compound of formula-4 comprising the steps of:
a) reacting the compound of formula-4 with an alkyl halide (RiX) to get a quaternary ammonium salt then with cytosine derivative of formula-5 to provide the compound of formula-6,

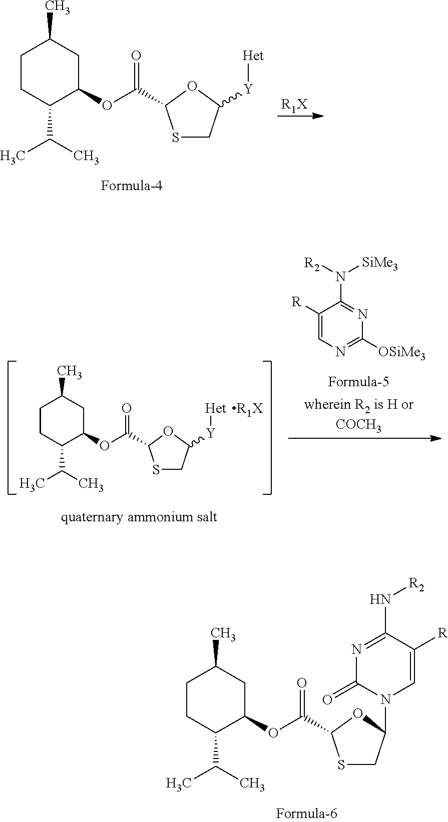

b) optionally de-protecting the compound of formula-6 to the compound of formula-7,

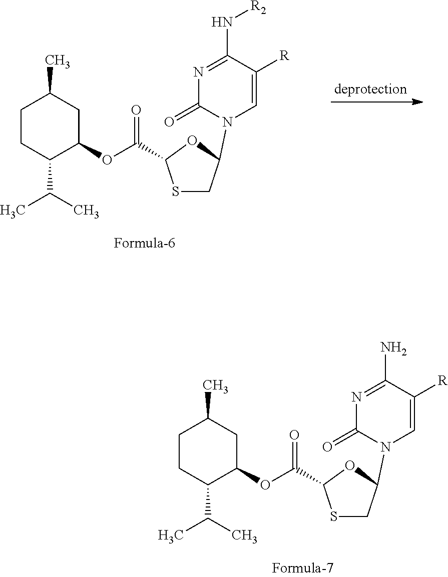

c) reducing compound of formula-7 with metal catalyst in presence of a buffer solution, then adding an organic acid to get the compound of formula-8.

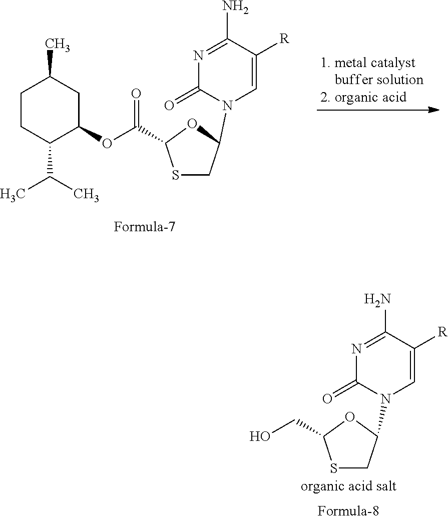

According to the present invention, compound of formula-4 is reacted with alkyl halide in a solvent or mixture thereof to get quaternary ammonium salt, which is in-situ reacted with cytosine derivative of formula-5 optionally in the presence of molecular sieves at 50-100° C. to get compound of formula-6.

The compound of formula-4 is dissolved in a solvent selected from toluene, acetone, dichloromethane, chloroform, dichloroethane, tetrahydrofuran, dimethylformamide, dimethyl sulfoxide or mixture thereof.

The alkyl halide used in the reaction is selected from methyl iodide, methyl bromide, ethyl iodide, ethyl bromide, butyl iodide, butyl bromide, trityl chloride, p-toluenesulphonyl chloride or methyl triflate.

According to the present invention, the compound of formula-6 is treated with an acid in a solvent at 10-40° C. for the deprotection and isolating the corresponding acid salt of formula-7. The obtained acid salt of formula-7 is reacted with a base in a solvent at 10-40° C. to get the compound of formula-7. However, the compound of formula-7 is also isolated in single step by treating the compound of formula-6 with an acid in a solvent at 10-40° C. followed by a base to adjust the pH of the reaction mass.

The solvents used for the dissolution of the compound of formula-6 or acid salt of formula-7 is selected from methanol, ethanol, isopropyl alcohol, n-butanol, iso-butanol, acetone, methyl isobutyl ketone, hexane, heptane, octane, ethyl acetate, propyl acetate, methyl acetate, tetrahydrofuran, dioxane, chloroform, dichloromethane, water or mixture thereof.

The acid used for the de-protection of the compound of formula-6 is selected from hydrochloric acid, sulfuric acid, methansulfonic acid, phosphoric acid, formic acid, acetic acid, benzenesulfonic acid or p-toluenesulfonic acid.

The base used for the reaction to adjust the pH of the reaction is selected from organic base such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

According to the present invention, compound of formula-7 is dissolved in a solvent or mixture thereof and reduced with metal catalyst at 15-45° C. in presence of a buffer solution to get crude cis-nucleoside derivative of formula-1. The crude cis-nucleoside derivative of formula-1 is treated with an organic acid at ambient temperature for 2-8 h to isolate cis-nucleoside derivative of formula-8, which is having less solubility as compared to the prior art acid salts. Hence, cis-nucleoside derivative of formula-8 is isolated with improved yield and quality.

The solvent used for the dissolution of compound of formula-7 is selected from ethanol, methanol, n-propanol, 2-propanol, N,N-dimethylformamide, tetrahydrofuran, water or mixture thereof.

The metal catalyst used for the reduction of the compound of formula-7 is selected from sodium borohydride, potassium borohydride, lithium borohydride or lithium aluminium hydride.

The buffer solution used in the reduction is selected from disodium hydrogen phosphate or dipotassium hydrogen orthophosphate.

The organic acid is selected from aromatic acids such as halobenzoic acids like 2-fluorobenzoic acid, 3-fluorobenzoic acid, 4-fluorobenzoic acid, 2-chlorobenzoic acid, 3-chlorobenzoic acid, 4-chlorobenzoic acid, 2-bromobenzoic acid, 3-bromobenzoic acid, 4-bromobenzoic acid, 2-iodobenzoic acid, 3-iodobenzoic acid or 4-iodobenzoic acid. Other organic acids includes 3-hydroxy-2-naphthoic acid, 2-methoxybenzoic acid, 3-methoxybenzoic acid, 4-methoxybenzoic acid, 2-methylbenzoic acid, 3-methylbenzoic acid, 4-methylbenzoic acid, 3-methylsalicylic acid, 4-methylsalicylic acid, 5-bromosalicylic acid, 3-methoxysalicylic acid, 4-methoxysalicylic acid or amino acids such as L-pyroglutamic acid or aspartic acid.

Yet another aspect of the present invention is to provide a novel compound of formula-4.

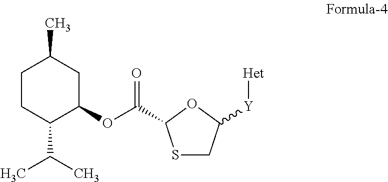

Yet another embodiment of the present invention is to provide a novel compound of formula-4a.

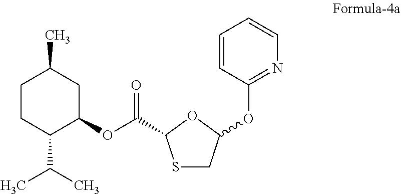

Yet another embodiment of the present invention is to provide a novel compound of formula-4b.

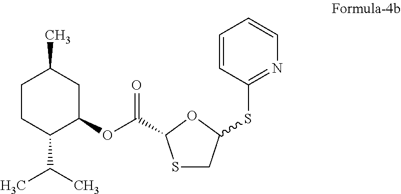

Yet another embodiment of the present invention is to provide a novel compound of formula-8a1.

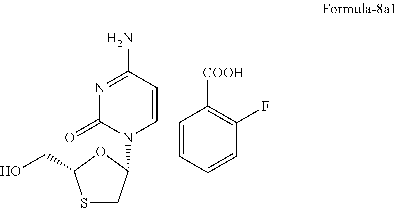

Yet another embodiment of the present invention is to provide a novel compound of formula-8b1.

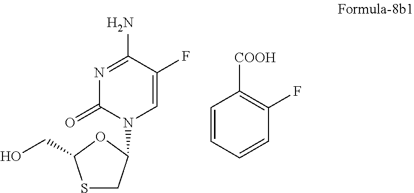

Yet another embodiment of the present invention is to provide a novel compound of formula-8b2.

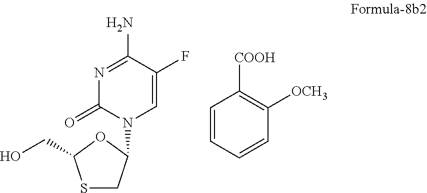

Yet another embodiment of the present invention is to provide a novel compound of formula 8b3.

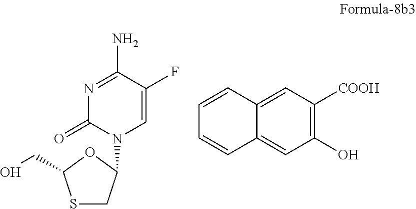

Yet another embodiment of the present invention is to provide a novel compound of formula-8b4.

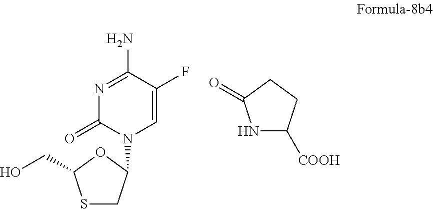

Yet another embodiment of the present invention is to provide pharmaceutical composition comprising: (a) a therapeutically effective amount of cis-nucleoside derivative of formula-1 or pharmaceutically acceptable salt; and (b) at least one pharmaceutically acceptable carrier.

The following examples are provided for illustrative purposes only and are not intended to limit the scope of the invention in any way.

### Powder X-Ray Diffraction (PXRD)

The X-ray diffraction patterns of said polymorphs of the invention were measured on Bruker D8 Discover powder diffractometer equipped with goniometer of θ/θ configuration and LynxEye detector. The Cu-anode X-ray tube was operated at 40 kV and 30 mA. The experiments were conducted over the 2θ range of 2.0°-50.0°, 0.030° step size and 50 seconds step time.

### Example-1

### Synthesis of 5-(pyridin-2-yloxy)-[1,3]oxathiolane-2-carboxylic acid 2-isopropyl-5-methyl-cyclohexyl ester (formula-4a)

### Step-1

To a solution of 5-hydroxy-[1,3]oxathiolane-2-carboxylic acid 2-isopropyl-5-methylcyclo-hexylester (formula-2, 100 g, 0.346 mol) in methylene dichloride (850 mL) containing methanesulphonic acid (0.45 g, 4.68 mmol) was added N,N-dimethylformamide (27.6 g, 0.38 mol) at 8-12° C. To the resultant reaction mass, thionyl chloride (44 g, 0.37 mol) was added at 8-12° C. and stirred for 2 h. The reaction mass was distilled to collect methylene dichloride (˜400 mL) and resultant mass was cooled to 22° C.

### Step-2

Triethylamine (32 g, 0.32 mol) was added to a solution of 2-hydroxypyridine (29.7 g, 0.313 mol) in methylene dichloride (100 mL) at 8-12° C. and raised the temperature to 40° C. The mixture of step-1 was added to the reaction mass over a period of 2 h. at 40-50° C. and maintained for 2-6 h. After completion of the reaction, the reaction mixture was subsequently washed with water, aq. sodium bicarbonate and water. The organic layer was evaporated to dryness by rotary evaporator, toluene (150 mL) was added to obtained residue and then distilled off to remove the traces of methylene dichloride. The resultant oily residue was diluted with toluene (400 mL) under stirring to get a uniform solution of compound of formula-4a.

¹H NMR (300 MHz, CDCl₃): δ 8.23-8.21 (m, 1H), 7.64-7.59 (m, 1H), 7.04-6.93 (m, 1H), 6.79-6.77 (d, J=8.28 Hz, 1H), 5.67 (s, 1H), 4.69-4.60 (m, 1H), 3.43-3.35 (m, 2H), 1.91-1.84 (m, 2H), 1.64-1.57 (m, 2H), 1.43-1.37 (m, 1H), 1.29-1.21 (m, 2H), 0.92-0.71 (m, 12H).

DIP MS: m/z (%) 366 (M+1)⁺.

### Example-2

### Synthesis of 5-(pyridin-2-ylsulfanyl)-[1,3]oxathiolane-2-carboxylicacid-2-isopropyl-5-methyl-cyclohexyl ester (Formula-4-b)

### Step-1

To a solution of 5-hydroxy-[1,3]oxathiolane-2-carboxylic acid 2-isopropyl-5-methylcyclo-hexylester (formula-2, 100 g, 0.346 mol) in methylene dichloride (850 mL) containing methanesulphonic acid (0.45 g, 4.68 mmol) was added N,N-dimethylformamide (27.6 g, 0.38 mol) at 8-12° C. To the resultant reaction mass, thionyl chloride (44 g, 0.37 mol) was added at 8-12° C. and stirred for 2 h. The reaction mass was distilled to collect methylene dichloride (˜400 mL) and resultant mass was cooled to 22° C.

### Step-2

To a solution of 2-mercaptopyridine (46.14 g, 0.415 mol) in dry acetone was added anhy. K₂CO₃ (62 g, 0.45 mol) and stirred at 40° C. for 30 min. Then 5-chloro-[1,3]oxathiolane-2-carboxylic acid 2-isopropyl-5-methyl-cyclohexylester of step-1 was added and stirred for 2 h. Progress of the reaction was monitored by HPLC. The reaction mass was diluted with DCM then subsequently washed with water, 1% KOH, water, dried and evaporated to get compound 4b.

¹H NMR (300 MHz, CDCl₃): δ 8.48-8.47 (d, J=4.13 Hz, 1H), 7.55-7.51 (m, 1H), 7.06-7.02 (m, 1H), 6.55-6.51 (m, 1H), 5.58 (s, 1H), 4.82-4.68 (m, 1H), 3.55-3.44 (m, 2H), 2.05-1.96 (m, 2H), 1.70-1.67 (m, 2H), 1.47-1.38 (m, 1H), 1.33-1.26 (m, 2H), 1.11-0.75 (m, 12H).

DIP MS: m/z (%) 382 (M+1)⁺.

### Example-3

### Synthesis of 5-(4-acetylamino-2-oxo-2H-pyrimidin-1-yl)-[1,3]oxathiolane-2-carboxylic acid 2-isopropyl-5-methyl-cyclohexyl ester (6a)

### Step-1

Ethyl iodide (83.8 g, 0.537 mol) and molecular sieves (4A, 12 g) were added to a solution of compound 4a in toluene (400 mL), stirred for 1 h and then filtered.

### Step-2

N-Acetyl cytosine (48.9 g, 0.32 mol), 1,1,1,3,3,3-hexamethyldisilazane (HMDS, 52.7 g, 0.326 mol) and methanesulfonic acid were added to toluene (125 mL), heated to reflux and was maintained for 3-4 h. The reaction mass was distilled completely and toluene (300 mL) was added to the obtained residue. Toluene (˜200 mL) was distilled out from the reaction mass and cooled to obtain the toluene solution of silylated acetyl cytosine of compound of formula-5a. The solution of step-1 was added to the reaction mass over a period of 2-2.5 h at 75-80° C. and maintained for 10 h. Sodium bicarbonate solution (54 g in 950 mL of water) was added and stirred for 4-5 h. The separated solid was filtered and subsequently washed with water, pre-cooled toluene and dried to get the title compound of formula-6a in 72 g.

### Example-4

### Synthesis of 5-(4-amino-2-oxo-2H-pyrimidin-1-yl)-[1,3]oxathiolane-2-carboxylic acid 2-isopropyl-5-methyl-cyclohexyl ester (7a)

The compound of formula-6a was suspended in methanol (20 mL) and methanesulphonic acid (2 g) was added. The resultant solution was stirred for 4 h. The reaction mixture was slowly added to a solution of DCM and aq NaHCO₃ solution carefully and stirred for 10 min. The organic layers were separated, washed with water dried and evaporated. The product was dissolved in ethyl acetate and precipitated by adding hexanes. The pure product was filtered and dried to get the compound of formula-7a.

### Example-5

### Synthesis of 5-(4-amino-2-oxo-2H-pyrimidin-1-[1,3]oxathiolane-2-carboxylic acid 2-isopropyl-5-methyl-cyclohexyl ester (7a)

The compound of formula-6a (100 g, 0.236 mol) was suspended in ethanol (550 mL) and methanesulphonic acid (57.4 g, 0.6 mol) was added. The resultant solution was stirred for 4 h. After completion of the reaction, hexane (1.1 L) was added to precipitate the product and stirred obtained slurry for 2 h. The separated solid was filtered, washed with a mixture of ethanol-hexane and dried to isolate methanesulphonic salt of compound of formula-7a.

The methanesulphonic acid salt of compound of formula-7a salt was suspended in a solvent mixture of ethyl acetate (250 mL)-hexane (150 mL) and treated with a solution of triethylamine (43.5 g) in hexane (100 mL). The reaction mixture was stirred for 1 h. Water (1 L) was added to the reaction mass and stirred for 60-90 min. The separated solid was filtered, washed with water and dried to isolate the compound of formula-7a in 65 g.

### Example-6

### Synthesis of 4-amino-1-(2R-hydroxymethyl-[1,3]-oxothiolane-5S-yl)1H-pyrimidin-3-one. 2-fluorobenzoic acid salt (formula-8a1)

Dipotassium hydrogen orthophosphate (83.3 g, 0.48 mol) was dissolved in a mixture of industrially methylated spirit (IMS, 600 mL) & purified water (200 mL) and the obtained solution was cooled to 18° C. The compound of formula-7a (100 g, 0.26 mol) was added at 15-22° C. and the suspension was stirred at 18-22° C. for 1 h. A solution of sodium borohydride (20.4 g, 0.54 mol) in water (110 mL) containing sodium hydroxide (40 mg)) was added drop wise by keeping temperature at 18-22° C. and maintained for 4 h. The completion of the reaction was confirmed by TLC. The reaction mass was transferred into a separating funnel and the layers were separated. The organic layer pH was adjusted to 5.9-6.3 with aq. HCl (˜25 mL) and readjusted to pH 7.5-7.8 with sodium hydroxide (15 mL, 15% w/w) and filtered. IMS (˜790 mL) was distilled out initially atmospherically followed by reduced pressure to reduce the traces of IMS. The resultant residue was diluted with water (200 mL) and then cooled to 22-30° C. Toluene (150 mL) was added to the reaction mass under stirring, allowed the layers to settle and separate the layers. Toluene layer was washed with water (100 mL) and combined aqueous layer was charcoalized. The filtrate was warmed to 38-42° C., 2-fluorobenzoic acid (37 g, 0.26 mol) was added and stirred for 2 h at the same temperature. The reaction mass was cooled to 22-30° C. and maintained for 3-4 h. The separated solid was filtered and washed with pre-cooled water and dried to get compound of formula-8a1 in 84 g.

Yield=83.3 g; SOR=−89° (c=1 in methanol); MR=128-132° C.; Purity: 99.92%

¹HNMR (300 MHz, DMSO-d₆): δ 12.89-13.51 (brs, 1H), 7.76-7.89 (m, 2H), 7.57-7.67 (m, 1H), 7.27-7.33 (m, 4H), 6.20 (t, 1H, J=5.2 Hz), 5.73 (d, 1H, J=7.4 Hz), 5.2-5.4 (brs, 1H), 5.17 (t, 1H, J=4.6 Hz), 3.67-3.74 (m, 2H), 3.37-3.43 (m, 1H), 3.04 (dd, 1H, J=11.68, 5.0 Hz).

### Example-7

### Process for the Preparation of Lamivudine Form-I

### Method-1

Lamivudine 2-fluorobenzoic acid salt (formula-8a1, 100 g, 0.27 mol) was taken into isopropyl alcohol (640 mL) containing water (22.5 mL) and a solution of triethylamine (45.8 g) in isopropyl alcohol (150 mL) was added to the reaction mass over a period of 3 h. at ambient temperature. The obtained slurry was cooled to 6-10° C. and maintained for 3 h. The separated product was filtered and washed with pre-cooled solution of isopropyl alcohol (4×45 mL). The wet cake was suspended into isopropyl alcohol (150 mL) containing water (1.5 mL) and triethylamine (1 g) and stirred for 2 h. The slurry was filtered and dried to isolate Lamivudine (52.0 g). The XRD, TGA & I.R analysis of the obtained product complies with Lamivudine polymorphic Form-I.

### Method-2

Lamivudine 2-fluorobenzoic acid salt (formula-8a1, 100 g, 0.27 mol) was taken into acetone (500 mL) containing 3% water (w/w) and a solution of triethylamine (52 g) in acetone (100 mL) was added to the reaction mass over a period of 40-60 min. at 50-60° C. under stirring and maintained for 15-20 min. The obtained slurry was cooled to 22-25° C. and maintained for 2 h. The separated product was filtered and washed with acetone (100 mL) containing 2% water (w/v). The wet product was dried at 40-46° C. under reduced pressure to obtain Lamivudine polymorphic form-I (55.0 g). The XRD, TGA & I.R analysis of the obtained product complies with Lamivudine polymorphic Form-I.

### Method-3

Lamivudine 2-fluorobenzoic acid salt (formula-8a1, 100 g, 0.27 mol) was taken into ethyl acetate (500 mL) containing 3% water (w/w). A solution of triethylamine (52 g, 0.51 mol) in ethyl acetate (100 mL) was added slowly a over period of 40-60 min. at 50-60° C. under stirring and maintained for 15-20 min. There after, the product slurry was cooled to 22-25° C. and maintained for 2 h. The separated product was filtered and washed with ethyl acetate (100 mL) containing 2% water (w/v). The wet product was dried at 40-46° C. under reduced pressure to obtain Lamivudine polymorphic form-I (55.2 g). The XRD, TGA & I.R analysis of the obtained product complies with Lamivudine polymorphic form-I.

### Method-4

Lamivudine 2-fluorobenzoic acid salt (formula-8a1, 100 g, 0.27 mol) was taken in to ethanol (500 mL) (containing 3% w/w water). A solution of triethylamine (52 g, 0.51 mol) in ethanol (100 mL) was added to reaction mass over a period of 40-60 min. at 50-60° C. under stirring and maintained for 15-20 min. There after, the product slurry was cooled to 22-25° C. and maintained for 2 h. The reaction mass further cooled to 6-8° C. and stirred for 2 h. The separated product was filtered and washed with cold ethanol (100 mL) containing 2% water (w/v). The wet product was dried at 40-46° C. under reduced pressure to obtain Lamivudine polymorphic form-I (49 g). The XRD, TGA & I.R analysis of the obtained product complies with Lamivudine polymorphic form-I.

### Example-8

### Synthesis of 5-(amino-2-oxo-2H-pyrimidin-1-yl)-[1,3]oxathiolane-2-carboxylic acid 2-isopropyl-5-methyl-cyclohexyl ester (7a)

To a solution of 2-mercaptopyridine (133 mg, 1.2 mmol) in dry acetone was added anhy. K₂CO₃ (1.3 mmol) and stirred at 40° C. for 30 min. Then 5-chloro-[1,3]oxathiolane-2-carboxylic acid 2-isopropyl-5-methyl-cyclohexylester (306 mg, 1.0 mmol) in DCM (20 mL) was added and stirred for 2 h. Progress of the reaction was monitored by HPLC. The reaction mass was diluted with DCM then subsequently washed with water, 1% KOH, water, dried and evaporated to get compound 4b.

The compound of formula-4b was dissolved in dry DCM (20 mL). 3% methyl iodide, silylated N-acetyl cytosine and powdered molecular sieves (4 A, 1 g) were added successively to the reaction mass, heated to reflux and maintained for 24 h. After the reaction was completed, the reaction mixture was cooled and filtered through celite. The reaction mixture was concentrated and the resultant residue was triturated with methanol. The obtained compound of formula-6a was filtered and dried.

The obtained compound of formula-6a was suspended in methanol (20 mL) and methane sulphonic acid (2 g) was added. The resultant solution was stirred for 4 h. The reaction mixture was slowly added to a solution of DCM and aq NaHCO₃ solution carefully and stirred for 10 min. The organic layers were separated, washed with water dried and evaporated. The product was dissolved in ethyl acetate and precipitated by adding hexanes. The pure product was filtered and dried to get the compound of formula-7a.

### Example-9

### Synthesis of 2R-(hydroxymethyl)-5S-cytosine-1′yl-1,3-oxathiolane (Lamivudine) from Lamivudine 3-hydroxy-2-naphthoic acid salt

Ethanol (600 mL) was added to a solution of dipotassium hydrogen orthophosphate (137 g in 220 mL of water) and the mass was cooled to 18° C. The compound of formula-7a (100 g, 0.26 mol) was added at 15-20° C. and the suspension was stirred at 18-20° C. for 1 hr. A solution of sodium borohydride (48 g in 95 mL of 0.12 N sodium hydroxide) was added drop wise by keeping temperature at 18-20° C. and maintained for 4 h. The completion of the reaction was confirmed by TLC. The reaction mass was transferred into a separating funnel and the layers were separated. The organic layer pH was adjusted to 6.0-6.5 with 6 N HCl (˜13 mL) and readjusted to pH 8.0 to 8.5 with 2N sodium hydroxide. Ethanol (˜790 mL) was distilled out under reduced pressure. The residue was cooled to 30-35° C., diluted with water (200 mL) and stirred for 15 min. Toluene (100 mL) was added to the reaction mass under stirring, allowed the layers to settle and separate the layers. Toluene layer washed with water (100 mL) and combined aqueous layer was charcoalised. 3-Hydroxy 2-naphthoic acid (48.92 g) and acetone (300 mL) were added to the aqueous layer and then heated to 60-65° C. to obtain clear solution. The reaction mass was cooled to 25-30° C. in 60 min to crystallize the material. Further, cooled to 10-15° C. and allowed to stir for 2 h. The separated solid was filtered and washed with water (50 mL) followed by pre-cooled acetone (10 mL). The material was dried under vacuum at 45-50° C. to yield Lamivudine 3-hydroxy-2-naphthoic acid salt in 90 g.

Lamivudine naphthylate (90 g) was suspended in 2% aqueous acetone (400 mL) and triethylamine (43.59 g) was added at 25-30° C. The reaction mass was heated to 40-45° C. and maintained for 30 min. The reaction mass was cooled to 25-30° C. over a period of 60 min. to crystallize the material. The separated solid was filtered, washed with acetone (20 mL) and dried under vacuum at 45-50° C. to obtained Lamivudine free base in 46 g.

### Example-10

### Synthesis of 5-(4-acetylamino-5-fluoro-2-oxo-2H-pyrimidin-1-yl)-[1,3]oxathiolane-2-carboxylic acid 2-isopropyl-5-methyl-cyclohexyl ester (6b)

### Step-1

To a solution of 5-hydroxy-[1,3]oxathiolane-2-carboxylic acid 2-isopropyl-5-methylcyclo-hexylester (formula-2, 100 g, 0.346 mol) in methylene dichloride (850 mL) containing methanesulphonic acid (0.45 g, 4.68 mmol) was added N,N-dimethylformamide (27.6 g, 0.38 mol) at 8-12° C. To the resultant reaction mass, thionyl chloride (44 g, 0.37 mol) was added at 8-12° C. and stirred for 2 h. The reaction mass was distilled to collect methylene dichloride (˜400 mL) and resultant mass was cooled to 22° C.

### Step-2

Triethylamine (32 g, 0.32 mol) was added to a solution of 2-hydroxypyridine (29.7 g, 0.313 mol) in methylene dichloride (100 mL) at 8-12° C. and raised the temperature to 40° C. The mixture of step-1 was added to the reaction mass over a period of 2 h at 40-50° C. and maintained for 2-6 h. After completion of the reaction, the reaction mixture was subsequently washed with water, aq. sodium bicarbonate and water. The organic layer was then evaporated to dryness by rotary evaporator and toluene (150 mL) was added and then distilled off to remove the traces of methylene dichloride. The resultant oily residue was diluted with toluene (400 mL) under stirring to get a uniform solution of compound of formula-4a.

### Step-3

Ethyl iodide (83.8 g, 0.537 mol) and molecular sieves (4A, 12 g) were added to a solution of compound 4a of step-2, stirred for 1 h and then filtered.

### Step-4

N-(5-Fluoro-2-oxo-1,2-dihydro-pyrimidin-4-yl)acetamide (54.72 g, 0.32 mol), 1,1,1,3,3,3-hexamethyldisilazane (HMDS, 52.7 g, 0.326 mol) and methanesulfonic acid were added to toluene (125 mL), heated to reflux and was maintained for 3-4 h. The reaction mass was distilled completely, toluene (300 mL) was added, distilled out toluene (˜200 mL) and cooled to the obtained the toluene solution of disilylated N-(5-fluoro-2-oxo-1,2-dihydro-pyrimidin-4-yl)-acetamide (formula-5b). The solution of step-1 was added to the reaction mass over a period of 2-2.5 h at 75-80° C. and maintained for 10 h. Sodium bicarbonate solution (54 g in 950 mL of water) was added and stirred for 4-5 h. The separated solid was filtered and washed subsequently washed with water and pre-cooled toluene and dried to get the title compound of formula-6b in 78 g.

### Example-11

### Synthesis of 5-(4-amino-5-fluoro-2-oxo-2H-pyrimidin-1-yl)-[1,3]oxathiolane-2-carboxylic acid 2-isopropyl-5-methylcyclohexyl ester (7b)

The compound of formula-6b was suspended in methanol (20 mL) and methanesulphonic acid (2 g) was added. The resultant solution was stirred for 4 h. The reaction mixture was slowly added to a solution of DCM and aq NaHCO₃ solution carefully and stirred for 10 min. The solvent was evaporated. The product was dissolved in ethyl acetate and precipitated by adding hexanes. The pure product was filtered and dried to get the compound of formula-7b.

### Example-12

### Synthesis of 5-(4-amino-5-fluoro-2-oxo-2H-pyrimidin-1-yl)-[1,3]oxathiolane-2-carboxylic acid 2-isopropyl-5-methyl-cyclohexyl ester (7b)

The compound of formula-6b (100 g, 0.236 mol) was suspended in ethanol (550 mL) and methanesulphonic acid (57.4 g, 0.6 mol) was added. The resultant solution was stirred for 4 h. After completion of the reaction, hexane (1.1 L) was added to precipitate the product and stirred obtained slurry for 2 h. The separated solid was filtered, washed with a mixture of ethanol-hexane and dried to isolate methanesulphonic salt of compound of formula-7b.

The methanesulphonic acid salt of compound of formula-7b salt was suspended in a solvent mixture of ethyl acetate (250 mL)-hexane (150 mL) and treated with a solution of triethylamine (43.5 g) in hexane (100 mL). The reaction mixture was stirred for 1 h. Water (1 L) was added to the reaction mass and stirred for 60-90 min. The separated solid was filtered, washed with water and dried to isolate the compound of formula-7b in 65 g.

### Example-13

### Synthesis of 4-amino-5-fluoro-1-(2R-hydroxymethyl-[1,3]-oxothiolane-5S-yl)1H-pyrimidin-3-one. 2-fluorobenzoic acid salt (formula-8b1)

Dipotassium hydrogen orthophosphate (83.3 g) was dissolved in a mixture of industrially methylated spirit (IMS, 600 mL) & purified water (200 mL) and the obtained solution was cooled to 18° C. The compound of formula-7b (100 g, 0.26 mol) was added at 15-22° C. and the suspension was stirred at 18-22° C. for 1 h. A solution of sodium borohydride (20.4 g (0.54 mol) in water (110 mL) containing sodium hydroxide (40 mg)) was added drop wise by keeping temperature at 18-22° C. and maintained for 4 h. The completion of the reaction was confirmed by TLC. The reaction mass was transferred into a separating funnel and the layers were separated. The organic layer pH was adjusted to 5.9-6.3 with aq. HCl (˜25 mL) and readjusted to pH 7.5-7.8 with sodium hydroxide (15 mL, 15% w/w) and filtered. IMS (˜790 mL) was distilled out initially atmospherically followed by reduced pressure to reduce the traces of IMS. The resultant residue was diluted with water (200 mL) and then cooled to 22-30° C. Toluene (150 mL) was added to the reaction mass under stirring, allowed the layers to settle and separate the layers. Toluene layer was washed with water (100 mL) and combined aqueous layer was charcoalized. The filtrate was warmed to 38-42° C., 2-fluorobenzoic acid (37 g, 0.26 mol) was added and stirred for 2 h at the same temperature. The reaction mass was cooled to 22-30° C. and maintained for 3-4 h. The separated solid was filtered and washed with pre-cooled water and dried to get compound of formula-8b1 in 80 g.

¹H NMR (300 MHz, DMSO-d₆): δ13.40 (brs, 1H), 8.20 (d, 1H, J=7.2 Hz), 7.84-8.00 (m, 2H), 7.58-7.68 (m, 2H), 7.28-7.34 (m, 2H), 6.12-6.16 (m, 1H), 5.41 (t, 1H, J=5.4 Hz), 5.18-5.20 (t, 1H, J=3.9 Hz), 3.70-3.82 (m, 2H), 3.39-3.45 (m, 1H), 3.09-3.15 (dd, 1H, J=11.85 & 4.35 Hz).

### Example-14

### Synthesis of Emtricitabine

The compound of formula-8b1 (100 g, 0.27 mol) was taken into isopropyl alcohol (640 mL) containing water (22.5 mL) and a solution of triethylamine (45.8 g) in isopropyl alcohol (150 mL) was added to the reaction mass over a period, of 3 h. at ambient temperature. The obtained slurry was cooled to 6-10° C. and maintained for 3 h. The separated product was filtered and washed with pre-cooled solution of isopropyl alcohol (4×45 mL). The wet cake was suspended into isopropyl alcohol (150 mL) containing water (1.5 mL) and triethylamine (1 g) and stirred for 2 h. The slurry was filtered and dried to isolate Emtricitabine (52.0 g).

### Example-15

### Process for the Preparation of Lamivudine Form-II

Lamivudine 2-fluorobenzoic acid salt (100 g) was taken in to ethanol (500 mL). A solution of triethylamine (52 g) in ethanol (100 mL) was added to the reaction mass over a period of 40-60 min. and maintained for 15-20 min. There after, the product slurry was cooled to 22-25° C. and maintained for 2 h. The reaction mass was distilled off to remove ethanol about half of the volume. The resultant reaction mass was diluted with ethyl acetate (200 mL) and stirred for 3 h. The product was filtered and washed with cold ethyl acetate (100 mL). The wet product was dried at 40-46° C. under reduced pressure to obtain Lamivudine polymorphic form-II (45 g). The XRD, TGA & I.R analysis complies with Lamivudine polymorphic form-II.

### Example-16

Brief manufacturing process for the pharmaceutical compositions comprising Lamivudine so prepared and an excipient/carrier is given below.

### Lamivudine Tablet

| | | mg/tablet | mg/tablet | mg/tablet | |
|---|---|---|---|---|---|
| S. No. | Ingredients | 100 mg | 150 mg | 300 mg | % w/w |
| 1. | Lamivudine | 100.00 | 150.000 | 300.000 | 50.00 |
| 2. | Microcrystalline Cellulose | 91.000 | 136.500 | 273.000 | 45.50 |
| 3. | Sodium Starch Glycolate | 8.000 | 12.000 | 24.000 | 4.00 |
| 4. | Magnesium Stearate | 1.000 | 1.500 | 3.000 | 0.50 |
| Core Tablet weight (mg) | | 200.00 | 300.000 | 600.000 | |
| 5. | Opadry ™ | — | 7.240 | 14.480 | NA |
| 6. | Opadry ™ | 4.827 | — | — | NA |
| 7. | Propylene Glycol | 0.173 | 0.260 | 0.520 | NA |
| 8. | Purified Water | q.s | q.s. | q.s. | NA |

| | | | |
|---|---|---|---|
| Coated Tablet weight (mg) | 205.000 | 307.500 | 307.500 |

1. Sift Lamivudine, Microcrystalline Cellulose and Sodium Starch Glycolate through Quadro Comil fitted with appropriate screen.
2. Sift Magnesium Stearate through Quadro Comil fitted with appropriate screen.
3. Load the material of step 1 in to Blender and blend. Add material of step 2 into it and blend.
4. Compress the tablet with appropriate tooling using Rotary Compression Machine.
5. Disperse Opadry White & Propylene Glycol in Purified Water with constant stirring to get a homogenous coating suspension.
6. Coat the core tablets of step 4 with coating suspension of step 5 using Coating Machine.

### Example-17

Brief Manufacturing Process for the composition of Lamivudine and Zidovudine using the Lamivudine so prepared is given below.

### Lamivudine and Zidovudine Tablet

| S. No. | Ingredients | mg/tablet | % w/w |
|---|---|---|---|
| 1. | Lamivudine | 150.000 | 20.00 |
| 2. | Zidovudine | 300.000 | 40.00 |
| 3. | Microcrystalline Cellulose | 258.750 | 34.50 |
| 4. | Colloidal Silicon Dioxide | 3.750 | 0.50 |
| 5. | Sodium Starch Glycolate | 30.000 | 4.00 |
| 6. | Magnesium Stearate | 7.500 | 1.00 |
| Core Tablet Weight (mg) | | 750.000 | — |
| 7. | Opadry ™ | 18.100 | NA |
| 8. | Propylene Glycol | 0.650 | NA |
| 9. | Purified Water | Qs | NA |

| | | |
|---|---|---|
| Coated Tablet Weight (mg) | 768.750 | — |

1. Sift Lamivudine, Zidovudine, and Microcrystalline Cellulose through Quadro Comill fitted with appropriate screen.
2. Sift Colloidal Silicon Dioxide and Sodium Starch Glycolate through Quadro Comill fitted with appropriate screen.
3. Sift Magnesium Stearate through Quadro Comill fitted with appropriate screen.
4. Load the material of step 1 and step 2 in to Blender and blend. Add material of step 3 and blend.
5. Compress the tablet with appropriate tooling using Rotary Tablet Compression Machine.
6. Disperse Opadry White and Propylene Glycol in Purified Water with constant stirring to get a homogenous coating suspension.
7. Coat the core tablets of step 5 with coating suspension of step 6 using Coating Machine

We claim:

1. A process for the preparation of cis-nucleoside derivative of Formula-1 comprising the steps of: 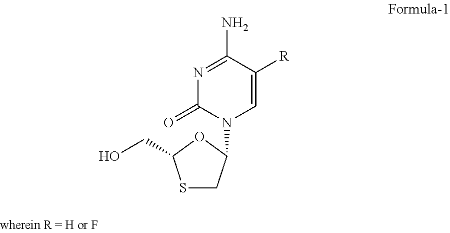
a) reacting the compound of formula-2 with chlorinating agent followed by compound of formula-3 in presence of a base to get compound of formula-4, 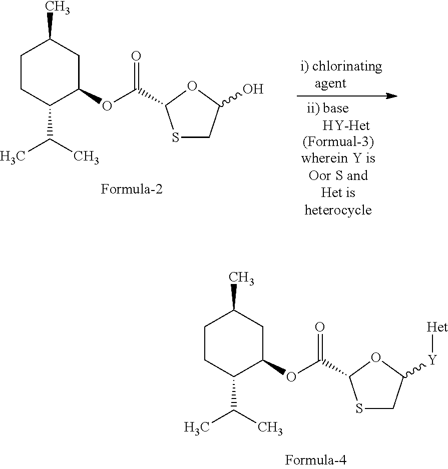
b) reacting the compound of formula-4 with an alkyl halide (R₁X) to get a quaternary ammonium salt and then with cytosine derivative of formula-5 to provide the compound of formula-6. 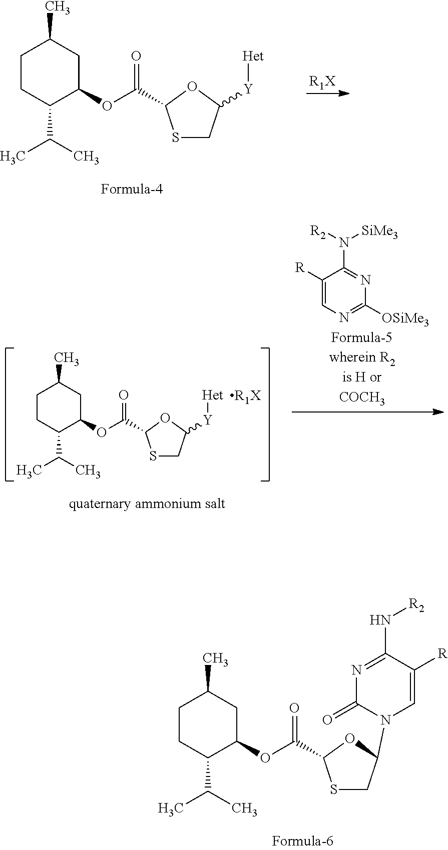
c) optionally de-protecting the compound of formula-6 to the compound of formula-7, 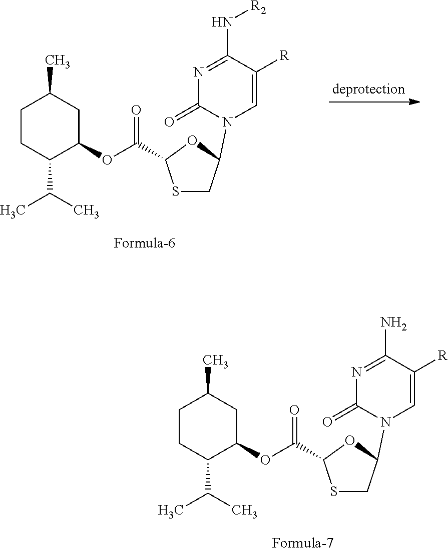
d) reducing compound of formula-7 with metal catalyst in presence of a buffer solution, then adding an organic acid to get the compound of formula-8, and 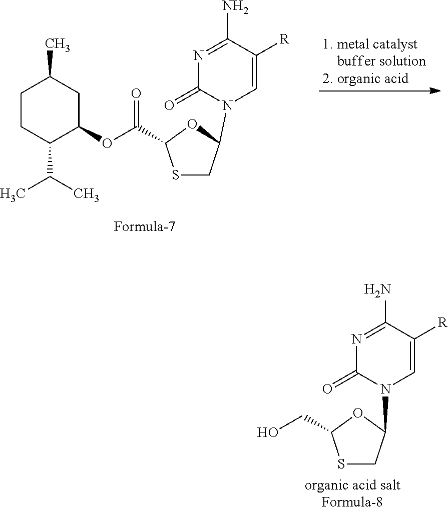
e) Converting the compound of formula-8 to cis-nucleoside derivative of formula-1.

2. A process for the preparation of Lamivudine comprising the steps of: 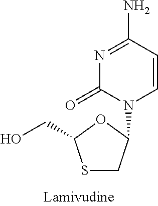
a) reacting the compound of formula-2 with chlorinating agent followed by compound of formula-3 in presence of a base to get compound of formula-4, 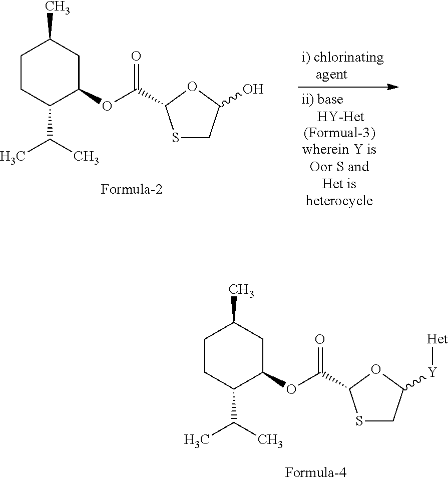
b) reacting the compound of formula-4 with an alkyl halide (R₁X) to get a quaternary ammonium salt and then with cytosine derivative of formula-5a to provide the compound of formula-6a, 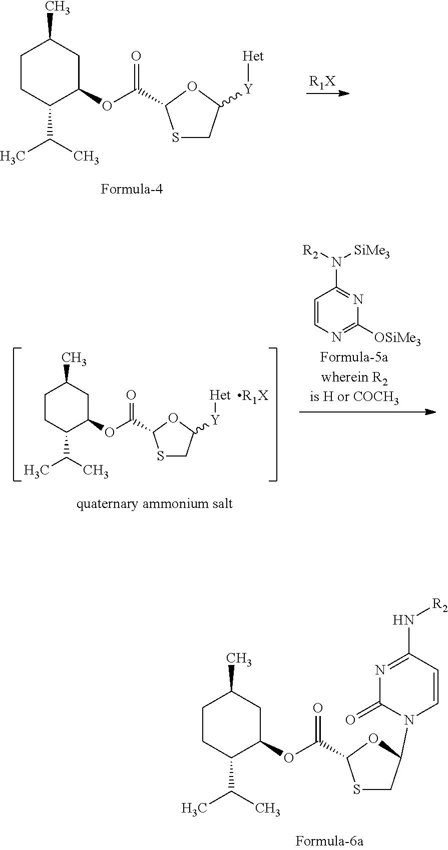
optionally de-protecting the compound of formula-6a to the compound of formula-7a, 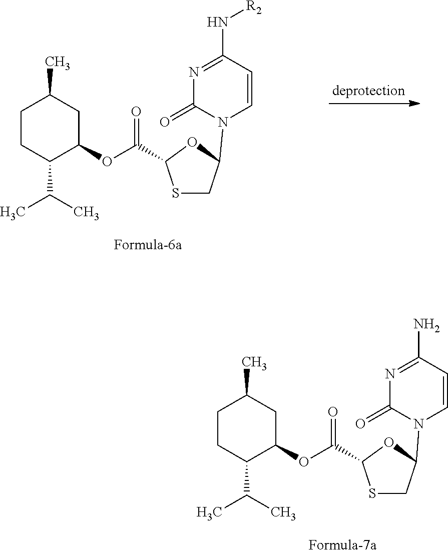
c) reducing compound of formula-7a with metal catalyst in presence of a buffer solution, then adding an organic acid to get the compound of formula-8a, and 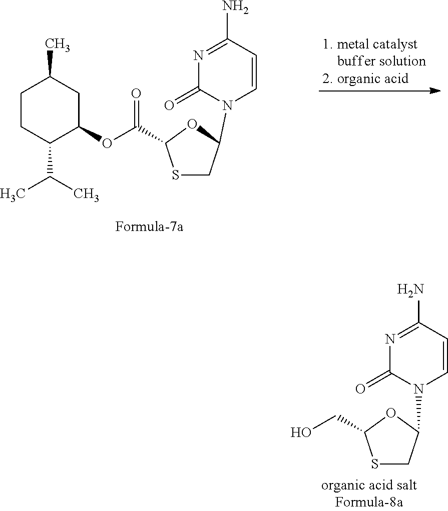
d) Converting the compound of formula-8a to Lamivudine.

3. A process for the preparation of Emtricitabine comprising the steps of: 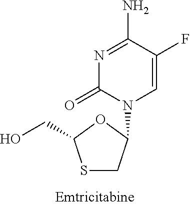
a) reacting the compound of formula-2 with chlorinating agent followed by compound Of formula-3 in presence of a base to get compound of formula-4, 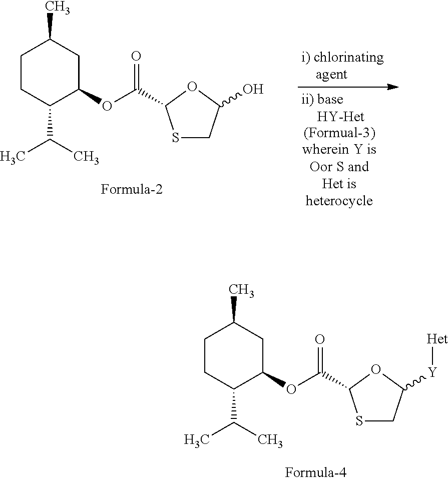
b) reacting the compound of formula-4 with an alkyl halide (R₁X) to get a quaternary, ammonium salt and then with cytosine derivative of formula-5b to provide the compound of formula-6b, 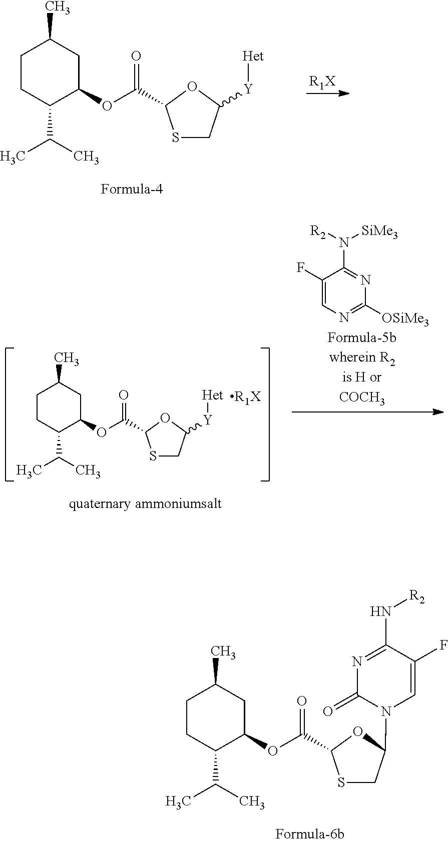
c) optionally de-protecting the compound of formula-6b to the compound of formula-7b, 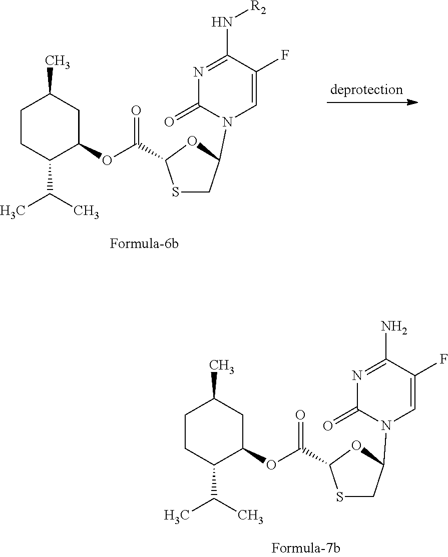
d) reducing compound of formula-7b with metal catalyst in presence of a buffer solution, then adding an organic acid to get the compound of formula-8b, and 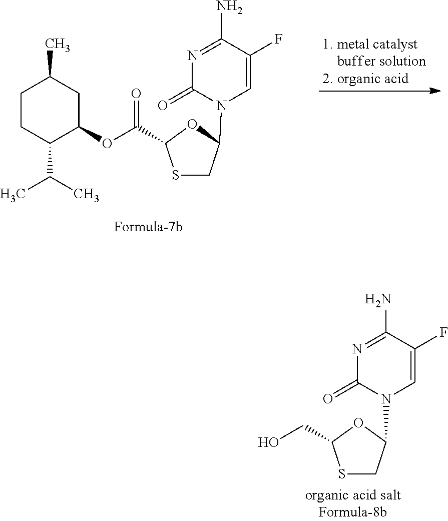
e) Converting the compound of formula-8b to Emtricitabine.

4. A process for the preparation of compound of formula-4 comprising the steps of:
a. reacting the compound of formula-2 with chlorinating agent followed by condensing with compound of formula-3 in presence of a base, and
b. isolating the compound of formula-4,

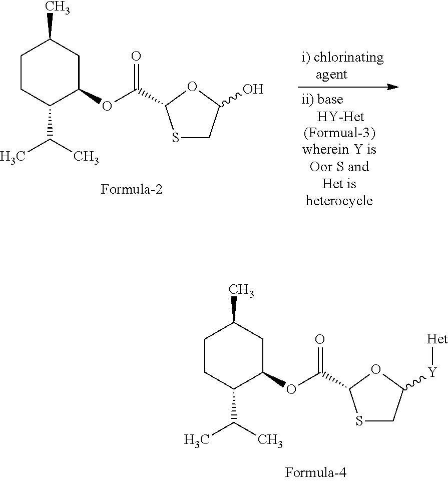

5. The process according to claims 1, 2, 3 and 4, wherein solvent used in the step a) is selected from dichloromethane, chloroform, dichloroethane, acetone, tetrahydrofuran, dimethylformamide, dimethyl sulphoxide or mixture thereof.

6. The process according to claims 1, 2, 3 and 4, wherein said chlorinating agent is selected from phosphorus pentachloride, phosphorus trichloride, thionyl chloride or triphenylphosphine dichloride.

7. The process according to claims 1, 2, 3 and 4, wherein said compound of formula-3 is selected from 2-mercaptopyridine, 4-mercaptopyridine, 2-hydroxypyridine, 4-hydroxypyridine, alkyl-2-mercaptopyridine, alkyl-4-mercaptopyridine, alkyl-2-hydroxypyridine, alkyl-4-hydroxypyridine, heteryl-2-mercaptopyridine, heteryl-4-mercaptopyridine, heteryl-2-hydroxypyridine, heteryl-4-hydroxypyridine, alkoxy-2-mercaptopyridine, alkoxy-4-mercaptopyridine, aryloxy-2-mercaptopyridine, aryloxy-4-mercaptopyridine, alkoxy-2-hydroxypyridine, alkoxy-4-hydroxypyridine, aryloxy-2-hydroxypyridine, aryloxy-4-hydroxypyridine, alkyloxycarbonyl-2-mercaptopyridine, alkyloxycarbonyl-4-mercaptopyridine, aryloxycarbonyl-2-mercaptopyridine, aryloxy-carbonyl-4-mercaptopyridine, alkyloxycarbonyl-2-hydroxypyridine, alkyloxycarbonyl-4-hydroxypyridine, aryloxycarbonyl-2-hydroxypyridine, aryloxycarbonyl-4-hydroxypyridine, 1,3-benzothiazol-2-ol, alkyl-1,3-benzothiazol-2-ol, alkoxy-1,3-benzothiazol-2-ol, 1,3-benzothiazol-2-thiol, alkyl-1,3-benzothiazol-2-thiol or alkoxy-1,3-benzothiazol-2-thiol.

8. The process according to claims 1, 2, 3 and 4, wherein said base used in the step a) is selected from organic bases such as triethylamine, tributylamine, N-methylmorpholine, N,N-diisopropylethylamine, N-methylpyrrolidine, pyridine, 4-(N,N-dimethylamino)pyridine, N-methylmorpholine, morpholine, imidazole, 2-methylimidazole, 4-methylimidazole and the like; inorganic bases such as alkali metal hydrides such as sodium hydride, potassium hydride and the like; sodamide; n-butyl lithium; lithium diisopropylamide; alkali metal hydroxides such as lithium hydroxide, sodium hydroxide, potassium hydroxide, cesium hydroxide; alkaline metal hydroxides such as aluminum hydroxide, magnesium hydroxide, calcium hydroxide and the like; alkali metal carbonates such as sodium carbonate, potassium carbonate, lithium carbonate, cesium carbonate and the like, alkaline earth metal carbonates such as magnesium carbonate, calcium carbonate and the like; alkali metal bicarbonates such as sodium bicarbonate, potassium bicarbonate and the like; ion exchange resins including resins bound to ions such as sodium, potassium, lithium, calcium, and magnesium, substituted or unsubstituted ammonium and the like; and other suitable bases.

9. The process according to claims 1, 2 and 3, wherein solvent used in the step b) is selected from toluene, acetone, dichloromethane, chloroform, dichloroethane, tetrahydrofuran, dimethylformamide, dimethyl sulfoxide or mixture thereof.

10. The process according to claims 1, 2 and 3, wherein said alkyl halide used in the step b) is selected from methyl iodide, methyl bromide, ethyl iodide, ethyl bromide, butyl iodide, butyl bromide, trityl chloride, p-toluenesulphonyl chloride or methyl triflate.

11. The process according to claims 1, 2 and 3, wherein solvent used for the deprotection in the step c) is selected from methanol, ethanol, isopropyl alcohol, n-butanol, iso-butanol, acetone, methyl isobutyl ketone, ethyl acetate, propyl acetate, methyl acetate, tetrahydrofuran, dioxane, chloroform, dichloromethane, water or mixture thereof.

12. The process according to claims 1, 2 and 3, wherein deprotection in the step c) is carried out with an acid selected from hydrochloric acid, sulfuric acid, methansulfonic acid, phosphoric acid, formic acid, acetic acid benzenesulfonic acid or p-toluenesulfonic acid.

13. The process according to claims 1, 2 and 3, wherein solvent used for the reduction in the step d) is selected from ethanol, methanol, n-propanol, 2-propanol, N,N-dimethylformamide, tetrahydrofuran, water or mixture thereof.

14. The process according to claims 1, 2 and 3, wherein said metal catalyst used for the reduction in the step d) is selected from sodium borohydride, potassium borohydride, lithium borohydride or lithium aluminium hydride.

15. The process according to claims 1, 2 and 3, wherein said buffer solution used for the reduction in the step d) is selected from disodium hydrogen phosphate or dipotassium hydrogen orthophosphate.

16. The process according to claims 1, 2 and 3, wherein said organic acid used for the saltification in the step d) is selected from aromatic acids such as halobenzoic acids like 2-fluorobenzoic acid, 3-fluorobenzoic acid, 4-fluorobenzoic acid, 2-chlorobenzoic acid, 3-chlorobenzoic acid, 4-chlorobenzoic acid, 2-bromobenzoic acid, 3-bromobenzoic acid, 4-bromobenzoic acid, 2-iodobenzoic acid, 3-iodobenzoic acid or 4-iodobenzoic acid. Other organic acids includes 3-hydroxy-2-naphthoic acid, 2-methoxybenzoic acid, 3-methoxybenzoic acid, 4-methoxybenzoic acid, 2-methylbenzoic acid, 3-methylbenzoic acid, 4-methylbenzoic acid, 3-methylsalicylic acid, 4-methylsalicylic acid, 5-bromosalicylic acid, 3-methoxysalicylic acid, 4-methoxysalicylic acid or amino acids such as L-pyroglutamic acid or aspartic acid.

17. The process according to claims 1, 2 and 3, wherein solvent used for the desaltification in the step e) is selected from methanol, ethanol, isopropyl alcohol, n-butanol, isobutanol, acetone, methyl isobutyl ketone, ethyl acetate, isopropyl acetate, tetrahydrofuran, dioxane, water or mixture thereof.

18. The process according to claims 1, 2 and 3, wherein base used for the desaltification in the step e) is selected from triethylamine, Hunig's base or ammonia.

19. A process for the preparation of compound of formula-1 comprising the steps of:
a. dissolving the compound of formula-8 in an organic solvent,
b. treating with a base, and
c. isolating the compound of formula-1.

20. The process according to claim 20, wherein said organic solvent is selected from methanol, ethanol, isopropyl alcohol, n-butanol, iso-butanol, acetone, methyl isobutyl ketone, ethyl acetate, isopropyl acetate, tetrahydrofuran, dioxane, water or mixture thereof.

21. The process according to claim 20, wherein said base is selected from triethylamine, Hunig's base or ammonia.

22. A compound of formula-8
Formula-8 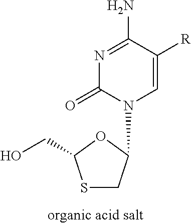
wherein

| Compound. No | R | Organic acid |
|---|---|---|
| 8a1 | H | 2-fluorobezoic acid |
| 8b1 | F | 2-fluorobezoic acid |
| 8b2 | F | 2-methoxy benzoaic acid |
| 8b3 | F | 3-hydroxynaphthalene-2-carboxylic acid |
| 8b4 | F | L-pyroglutamic acid |

23. A compound of formula-4

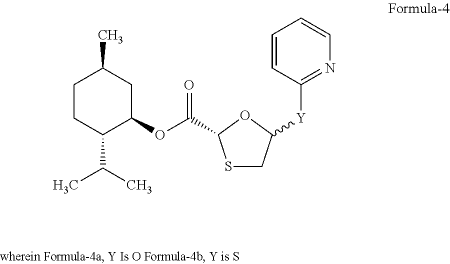

24. A pharmaceutical composition comprising: (a) a therapeutically effective amount of cis-nucleoside derivative of formula-1 or pharmaceutically acceptable salt; and (b) at least one pharmaceutically acceptable carrier.
